(19) United States
(12) Patent Application Publication
(10) Pub. No.: US 2023/0363337 A1
(43) Pub. Date: Nov. 16, 2023

(54) MELON WITH EXTENDED SHELF LIFE

(71) Applicant: VILMORIN & CIE, Paris (FR)

(72) Inventors: Fanny FOURCADE, Saint Remy de Provence (FR); Julie FAUVE, Chappes (FR)

(73) Assignee: VILMORIN & CIE, Paris (FR)

(21) Appl. No.: 18/029,755

(22) PCT Filed: Oct. 1, 2021
(86) PCT No.: PCT/EP2021/077065

§ 371 Date: Mar. 31, 2023

(30) Foreign Application Priority Data
Oct. 2, 2020 (WO) PCT/IB2020/000819

(51) Int. Cl. A01H 6/34; A01H 1/04; A01H 5/08

(52) U.S. Cl. CPC A01H 6/344; A01H 1/045; A01H 5/08

(57) ABSTRACT

A Cucumis melo plant, wherein the plant homozygously includes in its genome a mutant allele of the staygreen (sgr) gene on chromosome 9, wherein the mutant allele of the sgr gene includes at least one loss-of-function mutation in comparison to the sequence of a wild-type sgr allele (SEQ ID NO:1) and wherein the mutant allele of the sgr gene confers rind color stability to the fruits of the plant at maturity and/or during post-harvest, in comparison with an isogenic non-long shelf life (non-LSL) Cucumis melo plant which does not include the mutant allele. This further relates to parts, cells and seeds of the plants, as well as related methods and processes.

The present invention relates to Cucumis melo (C. melo) plants with an extended shelf life in comparison with existing non-Long Shelf Life (non-LSL) types of melon, whilst their properties such as sugar content, cycle length, aroma or firmness remain similar to non-LSL melons. The present invention also provides methods of making such plants, and methods of detecting and/or selecting such plants.

Melon (Cucumis melo L.) is a worldwide grown crop from the family Cucurbitaceae. Most of the commercial melons produce sweet fruits known for example as Charentais, Cantaloupe, Piel de sapo, Galia, Ananas, Honeydew. Melon fruits are usually consumed as dessert fruits.

Combining a long shelf life with a desirable taste for the consumer has always been a challenge for melon breeders. On the one hand, shelf life is an important parameter for melon growers and retailers. Fruits with extended shelf life can be stored during longer period of times, thus reducing commercial losses, and providing an increased flexibility in harvest and transport. Many efforts have been made to improve the shelf life of melons. Up to the 1980s, marketed melons were mainly traditional types of melons, with a limited shelf life. Traditional melons are climacteric fruits in which the ripening process is triggered by a burst of ethylene production, accompanied by a development of respiration. In turn, these events trigger a number of ethylene-dependent processes, such as a change of rind color, generally a yellowing, the development of aroma giving taste to the melons, or a gradual softening of the fruits. These processes have an influence on the shelf life of melon.

During the 1990s, long shelf life (LSL) types of melons have been introduced and progressively represented a large part of the market. LSL melons are non-climacteric melons which do not produce the ethylene burst which is typical of climacteric melons, at maturity. Moreover, LSL melons keep their green colour for a longer period of time, and remain firm after harvest. Whilst the extended shelf life provides important advantages in comparison with the traditional melons, LSL melons also have significant drawback: they develop less aroma, such that their flavor is often perceived negatively by the consumer. Candidate mutations have been identified, which could be responsible for the extended shelf life of LSL melons, in particular in the ACC oxidase gene (Ayub, Ricardo, et al. “Expression of ACC oxidase antisense gene inhibits ripening of cantaloupe melon fruits.” Nature biotechnology 14.7 (1996): 862-866)

More recently, intermediate shelf life (ISL) melons were obtained and marketed, as an attempt to provide melons with a better taste than LSL melons whilst offering an acceptable shelf life. However such compromise between complex traits is a difficult task for breeders to achieve. There remains a need to provide new melon typologies meeting the grower and consumer's demand, combining an improved shelf life with a good taste and other commercially important properties.

### SUMMARY OF THE INVENTION

The inventors of the present application have discovered that inactivating the staygreen (sgr) gene on chromosome 9, e.g. by a splicing site mutation, conferred an increased rind color stability at maturity and during post-harvest, to non-LSL melon types, whilst no effect on rind color is observed on LSL melons. This stability, in turn is associated with an increased shelf life of the fruits. This was surprising since other genes, such as the ACC oxidase (Ayub, Ricardo, et al., 1996), have been previously involved in the control of the shelf life of melon, but not the sgr gene. Surprisingly, the inventors have also discovered that the sgr mutants had a number of other advantageous properties which remain the same or comparable to non-LSL melons such as traditional melons. These properties have some interest for the grower, retailer or consumer: the cycle length, the firmness, the soluble solid content or brix (i.e. the sweetness), or the rate of peduncle abscission. The invention thus provides new typologies of melon, combining an extended shelf life with non-LSL properties of commercial interest such as cycle length, sweetness, peduncle abscission and softening at maturity.

Accordingly, in one aspect, the present invention relates to a Cucumis melo plant, wherein said plant homozygously comprises in its genome a mutant allele of the staygreen (sgr) gene on chromosome 9, wherein said mutant allele of the sgr gene comprises at least one loss-of-function mutation in comparison to the sequence of a wild-type sgr allele (SEQ ID NO:1) and wherein said mutant allele of the sgr gene confers rind color stability to the fruits of said plant at maturity and/or during post-harvest, in comparison with an isogenic non-long shelf life (non-25 LSL) Cucumis melo plant which does not comprise said mutant allele at a homozygous state, and which thus comprises a functional sgr gene, heterozygously or homozygously.

Another object of the invention relates to a cell of a C. melo plant according the invention, preferably a cell derived from an embryo, protoplast, meristematic cell, callus, pollen, leaf, anther, stem, petiole, root, root tip, fruit, seed, flower, cotyledon, and/or hypocotyl, wherein said cell homozygously comprises in its genome a mutant allele of the staygreen (sgr) gene on chromosome 9, wherein said mutant allele of the sgr gene comprises at least one loss-of-function mutation in comparison to the sequence of a wild-type sgr allele (SEQ ID NO:1).

The invention also relates to a plant part of a C. melo plant comprising at least one cell according to the invention, preferably an embryo, protoplast, meristematic cell, callus, pollen, leaf, anther, stem, petiole, root, root tip, fruit, seed, flower, cotyledon, and/or hypocotyl, in particular a fruit.

The invention further relates to a C. melo seed, which can be grown into a C. melo plant according to the invention.

In a further aspect, the invention relates to an in vitro cell or tissue culture of regenerable cells of the C. melo plant according to the invention, wherein the regenerable cells are derived from an embryo, protoplast, meristematic cell, callus, pollen, leave, anther, stem, petiole, root, root tip, seed, flower, cotyledon, and/or hypocotyl.

The invention also relates to a method of producing a C. melo plant producing fruits or susceptible to produce fruits with an increased shelf life, comprising
(a) obtaining a part of a plant according to the invention,
(b) vegetatively propagating said plant part to generate a plant from said plant part.

The invention further relates to a method of producing a C. melo plant producing fruits or susceptible to produce fruits with an increased shelf life, comprising the introduction of a loss-of-function mutation in the sgr gene on chromosome 9 (SEQ ID NO:1), in the genome of a non-LSL C. melo plant, wherein said mutation is introduced by mutagenesis or genome editing, in particular by a technique selected from ethyl methanesulfonate (EMS) mutagenesis, oligonucleotide directed mutagenesis (ODM), Zinc finger nuclease (ZFN) technology, Transcription Activator-Like Effector Nucleases (TALENs) the CRISPR/Cas system, engineered meganuclease, re-engineered homing endonucleases and DNA guided genome editing.

Further provided is a method for identifying, detecting and/or selecting C. melo plants producing fruits or susceptible to produce fruits with an increased shelf life, said method comprising the detection of a mutant allele of the sgr gene on chromosome 9 in the genome of said plants, wherein said mutant allele comprises at least one loss-of-function mutation in comparison to the sequence of a wild-type sgr allele (SEQ ID NO:1).

The invention further relates to a method for improving the shelf life of melon fruit, the marketability of melon fruit and/or the yield of melon production, wherein said method comprises growing C. melo plants according to the invention and harvesting fruits set by said plants.

Also provided is a method of producing melon fruit comprising:
a) growing a C. melo plant according to the invention;
b) allowing said plant to set fruit; and
c) harvesting fruit of said plant, preferably at pre-mature or mature stage.

Another object of the invention is the use of a C. melo plant according to the invention or a fruit thereof in the fresh cut market or for food processing.

### DEFINITIONS

Melon types can be classified according to their postharvest characteristics into three groups: traditional, intermediate shelf life (ISL) and long shelf-life (LSL).

The term “shelf life” herein relates to the period of time during which a melon fruit can be stored post-harvest before it is considered unsuitable for sale or consumption. Shelf life is preferably assessed during storage. Shelf life takes generally account of various characteristics of the fruit, such as the rind color, the flesh color, the firmness, the aroma and/or sugar content. Preferably, the increased shelf life of the melons according to the invention is assessed on the basis of an improved color stability at harvest and/or during postharvest storage. In particular, the melons according to the invention retain their immature color for a longer period of time during postharvest storage, compared to melons that do not have the genetic features of the present invention.

“Long shelf life (LSL)” melons typically have shelf lives of at least 10 days, preferably at least 14 days. More particularly, a LSL melon has a shelf life between 10 and 21 days. LSL melons are non-climacteric. In particular, a LSL melon may be selected from the following types: LSL Charentais, LSL Italian netted, Harper, LSL Galia, Yellow Canary, Piel de Sapo and Honey Dew.

“Traditional” melons typically have shelf lives of less than 5 days. Preferably, a traditional melon has a shelf life between 2 and 5 days. Typically, traditional melons are climacteric. In particular, a traditional melon may be selected from the following types: traditional Charentais, traditional Italian netted, Western Shipper, Eastern Shipper, traditional Galia, traditional Ananas.

“Intermediate shelf life (ISL)” melon are melons with a shelf life between the shelf life of a traditional melon and the shelf life of a LSL melon. Preferably, an ISL melon has a shelf life between 7 to 14 days. In particular, an ISL melon may be selected from the following types: Charentais, Italian netted, Western Shipper, Eastern Shipper, Galia, Ananas and Honey Dew.

As used herein, the terms “non-LSL” melon relate to a traditional or ISL melon. Any reference to non-LSL melons or C. melo plants is thus to be understood as designating traditional and/or ISL melons or C. melo plants. Non-LSL melons typically have a shelf life of less than 14 days and preferably less than 10 days. In particular, a non-LSL melon may be selected from the following types: traditional Charentais, traditional Italian netted, traditional Galia, traditional Ananas, ISL Charentais, ISL Italian netted, Western Shipper, Eastern Shipper, ISL Galia, ISL Ananas and ISL Honey Dew.

“Climacteric” types of melon are characterized by the rapid, autocatalytic production of ethylene at maturity, generally concomitant with an increase in respiration. Climacteric ripening is accompanied by several ethylene mediated physiological and biochemical events such as flesh softening, aroma production, rapid rind color change and abscission from the vine (i.e. peduncle abscission). The rind change color varies depending on the type of melon. In Galia-type melons, the fruit rinds change from dark green to yellow-orange whilst rinds of Charentais-type melons change from green or grey to creamy yellow. The autocatalytic production of ethylene manifests as exponentially increasing concentrations of ethylene over time in the cavity of the melon, generally progressing from negligible to a maximum over just a few days. The absolute magnitude of the peak level of ethylene varies among climacteric melon cultivars, however the rapid induction of ethylene biosynthesis is characteristic of such lines.

Non-climacteric types of melon do not exhibit such autocatalytic production of ethylene, and are therefore characterized by a reduced rind color change at maturity or no color change at all, by the retention of firmness during storage and a decreased production of aroma, which has a negative impact on the flavor of such melons.

As used herein, an “allele” refers to any of several alternative or variant forms of a genetic unit, such as a gene, which are alternative in inheritance because they are positioned at the same locus in homologous chromosomes. Such alternative or variant forms may be the result of single nucleotide polymorphisms, insertions, inversions, translocations or deletions, or the consequence of gene regulation caused by, for example, by chemical or structural modification, transcription regulation or post-translational modification/regulation. In a diploid cell or organism, the two alleles of a given gene or genetic element typically occupy corresponding loci on a pair of homologous chromosomes.

As used herein, the term “cross”, “crossing”, “cross pollination” or “cross-breeding” refer to the process by which the pollen of one flower on one plant is applied (artificially or naturally) to the ovule (stigma) of a flower on another plant.

As used herein, the term “genotype” refers to the genetic makeup of an individual cell, cell culture, tissue, organism (e.g., a plant), or group of organisms.

As used herein, the term “heterozygote” refers to a diploid or polyploid individual cell or plant having different alleles (forms of a given gene, genetic determinant or sequences) present at least at one locus.

As used herein, the term “heterozygous” refers to the presence of different alleles (forms of a given gene, genetic determinant or sequences) at a particular locus.

As used herein, “homologous chromosomes”, or “homologs” (or homologues), refer to a set of one maternal and one paternal chromosomes that pair up with each other during meiosis. These copies have the same genes in the same loci and the same centromere location.

As used herein, the term “homozygote” refers to an individual cell, or plant having the same alleles at one or more loci on all homologous chromosomes.

As used herein, the term “homozygous” refers to the presence of identical alleles at one or more loci in homologous chromosomal segments. Accordingly, a plant which homozygously comprises in its genome a mutant allele of the staygreen (sgr) gene on chromosome 9, comprises said mutant allele in all copies of the sgr gene on chromosome 9, for instance two copies if the plant is diploid and comprises a set of two homologous chromosomes 9.

As used herein, the term “hybrid” refers to any individual cell, tissue, plant part or plant resulting from a cross between parents that differ in one or more genes. An F1 hybrid (HF1) results from the cross of two genetically different parent cultivars or lines. Hybrid plants according to the invention are heterozygous for one or several genes in their genome, but are homozygous for the sgr gene, i.e. all of their sgr alleles (i.e. 2 for a diploid plant) are loss-of-function mutant alleles. The loss-of-function mutation may be or not be the same in every sgr allele. Example 2 and FIG. 1 describe techniques for the generation of HF1 plants homozygously comprising sgr mutant alleles.

As used herein, two plants are said “isogenic”, when they have the same or essentially the same set of chromosomes and genes, except for one gene, in the present invention the sgr gene. The two isogenic plants thus comprise different alleles of the sgr gene. Comparing the phenotype of two isogenic plants allows the evaluation of the effect of an allelic variation of the sgr gene.

As used herein, a “loss-of-function mutation”, or “inactivating mutation”, is a mutation which results in the gene product having a reduced function or no function at all (being partially or wholly inactivated). When the allele has a complete loss of function, it is also called a null allele. Phenotypes associated with such mutations are generally recessive.

As used herein, the terms “molecular marker” refer to an indicator that is used in methods for visualizing differences in characteristics of nucleic acid sequences. Examples of such indicators are restriction fragment length polymorphism (RFLP) markers, amplification fragment length polymorphism (AFLP) markers, single nucleotide polymorphisms (SNPs), insertion mutations, microsatellite markers (SSRs), sequence-characterized amplified regions (SCARs), cleaved amplified polymorphic sequence (CAPS) markers or isozyme markers or combinations of the markers described herein which defines a specific genetic and chromosomal location. Mapping of molecular markers in the vicinity of an allele is a procedure which can be performed quite easily by the person skilled in the art using common molecular techniques.

As used herein, the term “primer” refers to an oligonucleotide which is capable of annealing to the amplification target allowing a DNA polymerase to attach, thereby serving as a point of initiation of DNA synthesis when placed under conditions in which synthesis of primers extension product is induced, i.e., in the presence of nucleotides and an agent for polymerization such as DNA polymerase and at a suitable temperature and pH. The primer is preferably single stranded for maximum efficiency in amplification. Preferably, the primer is an oligodeoxyribonucleotide. The primer must be sufficiently long to prime the synthesis of extension products in the presence of the agent for polymerization. The exact length of the primers will depend on many factors, including temperature and composition (A/T and G/C content) of primer. A pair of bi-directional primers consists of one forward and one reverse primer as commonly used in the art of DNA amplification such as in PCR amplification.

As used herein, a single nucleotide polymorphism (SNP) is a DNA sequence variation occurring when a single nucleotide—A, T, C, or G—in the genome (or other shared sequence) differs between members of a biological species or paired chromosomes in an individual. For example, two sequenced DNA fragments from different individuals, AAGCCTA to AAGCTTA, contain a difference in a single nucleotide. In this case there are two alleles: C and T.

As used herein, “marker-based selection” or “marker-assisted selection (MAS)” or “marker-assisted breeding (MAB)” refers to the use of genetic markers to detect one or more nucleic acids from a plant, wherein the nucleic acid is associated with a desired trait to identity plants that carry genes for desirable (or undesirable) traits, so that those plants can be used (or avoided) in a selective breeding program.

As used herein, “maturity” is a stage of melon fruit development. Senescence of the first leave and of the fruit tendril are maturity indicator common to climacteric and non-climacteric melons. Additional maturity indicators for climacteric melons are the peduncle cracking or the release of aroma. In non-climacteric types of melons, such as Piel de Sapo or Yellow Canary, maturity is indicated by the browning or yellowing of the pistillate area as well as a coloration progressing to the peduncle.

As used herein, the term “offspring” or “progeny” refers to any plant resulting as progeny from a vegetative or sexual reproduction from one or more parent plants or descendants thereof. For instance an offspring plant may be obtained by cloning or selfing of a parent plant or by crossing two parents plants and include selfings as well as the F1 or F2 or still further generations. An F1 is a first-generation offspring produced from parents at least one of which is used for the first time as donor of a trait, while offspring of second generation (F2) or subsequent generations (F3, F4, etc.) are specimens produced from selfings of F1's, F2's etc. An F1 may thus be (and usually is) a hybrid resulting from a cross between two true breeding parents (true-breeding is homozygous for a trait), while an F2 may be (and usually is) an offspring resulting from self-pollination of said F1 hybrids.

As used herein, the term “melon” means any type, variety, cultivar of the Cucumis melo species. The invention encompasses plants of different ploidy levels, whether a diploid plant, but also a triploid plant, a tetraploid plant, etc.

As used herein, the term “plant part” refers to any part of a plant including but not limited to the shoot, root, stem, seeds, fruits, leaves, petals, flowers, ovules, branches, petioles, internodes, pollen, stamen, rootstock, scion and the like.

The term “resistance” is as defined by the ISF (International Seed Federation) Vegetable and Ornamental Crops Section for describing the reaction of plants to pests or pathogens, and abiotic stresses for the Vegetable Seed Industry. Specifically, by resistance, it is meant the ability of a plant variety to restrict the growth and development of a specified pest or pathogen and/or the damage they cause when compared to susceptible plant varieties under similar environmental conditions and pest or pathogen pressure. Resistant varieties may exhibit some disease symptoms or damage under heavy pest or pathogen pressure.

As used herein, the term “susceptible” refers to a plant that is unable to restrict the growth and development of a specified pest or pathogen.

As used herein, the term “inbred” or “line” refers to a relatively true-breeding strain.

As used herein, the term “phenotype” refers to the observable characters of an individual cell, cell culture, organism (e.g. a plant), or group of organisms which results from the interaction between that individual genetic makeup (i.e. genotype) and the environment.

As used herein, the terms “introgression”, “introgressed” and “introgressing” refer to the process whereby genes of one species, variety or cultivar are moved into the genome of another species, variety or cultivar, by crossing those species. The crossing may be natural or artificial. The process may be optionally be completed by backcrossing to the recurrent parent, in which case introgression refers to infiltration of the genes of one species into the gene pool of another through repeated backcrossing of an interspecific hybrid with one of its parents. An introgression may be also described as a heterologous genetic material stably integrated in the genome of a recipient plant.

In the present specification, a comparison between two or more melon plants or fruits, in particular a comparison between a melon plant according to the invention with an isogenic melon not comprising a mutant allele of the sgr gene on chromosome 9, is understood to be a comparison between plants or fruits at the same stage of maturity or at the same stage post-harvest, grown in the same environmental conditions.

### SEQUENCE LISTING

SEQ ID NO :1 shows the sequence of a wild-type sgr gene on chromosome 9.
SEQ ID NO: 2 shows the sequence of the sgr-1 allele of the sgr gene, comprising a G584A mutation
SEQ ID NO: 3 shows the coding sequence of a wild-type sgr gene on chromosome 9.
SEQ ID NO: 4 shows the amino acid sequence of a wild-type SGR protein.
SEQ ID NO:5 shows a context sequence for the development of markers around the sgr-1 mutation
SEQ ID NO:6 shows a sequence of a forward primer for detecting a wild-type allele of the sgr gene.
SEQ ID NO:7 shows a sequence of a forward primer for detecting the sgr-1 mutant allele of the sgr gene.
SEQ ID NO:8 shows a sequence of a common reverse primer for detecting the sgr-1 and wild-type mutant allele of the sgr gene.

### LEGEND OF THE FIGURES

FIG. 1 shows a breeding scheme for introducing the sgr-1 mutation in HF1 hybrids.

FIG. 2 shows pictures of leaves of Charentais, Yellow Canary and Galia melons, either wild-type allele or comprising the sgr-1 mutation.

FIG. 3 shows a picture of Italian netted melon leaves, either wild-type allele or comprising the sgr-1 mutation, under CYSDV pressure.

FIG. 4 shows the L*, a* and b* values of leaf color of the varieties V1_Charentais and V2-Yellow Canary, either wild-type allele or comprising the sgr-1 mutation.

FIG. 5 shows the evolution of the ΔE* value of leaf color at three dates, wherein the ΔE* value reflects the leaf color difference in the CIELAB color space between sgr-1 mutant melons and the corresponding wild-type (WT) melons for the varieties V1_Charentais and V2-Yellow Canary.

FIG. 6 shows photographs of the rind fruits of variety V2-ItalianNet_NLSL after 7 days of storage, either WT (Panel A) or with the sgr-1 mutation (Panel B).

FIG. 7 shows the L*, a* and b* values (from left to right for each genotype) of the rind color of varieties V1_Charentais_LSL, V2_ItalianNet_NLSL and V3_ItalianNet_NLSL, on the day of harvest (upper panel) or after 7 days of storage (lower panel).

FIG. 8 shows the ΔE* value at two dates (day of harvest, and after 7 days of storage from left to right) of varieties V1_Charentais_LSL, V2_ItalianNet_NLSL and V3_ItalianNet_NLSL, wherein the ΔE value reflects the rind color difference in the CIELAB color space between sgr-1 mutant melons and the corresponding wild-type (WT) melons.

FIG. 9 shows the L*, a* and b* values (from left to right for each genotype) of the flesh color of the varieties V1_Charentais_LSL, V2_ItalianNet_NLSL and V6_YellowC_LSL, either wild-type allele or comprising the sgr-1 mutation, after 7 days of storage.

FIG. 10 shows an assessment of the cycle length of different melon varieties (V2_ItalianNet_NLSL, V4_HD_NLSL and V5_Charentais_NLSL), either comprising the sgr-1 mutation or wild-type allele.

FIG. 11 shows an assessment of peduncle abscission for different melon genotypes (V2_ItalianNet_NLSL, V4_HD_NLSL and V5_Charentais_NLSL), either comprising the sgr-1 mutation or wild-type allele.

FIG. 12 shows a measurement of the brix for different melon genotypes (V2_ItalianNet_NLSL, V4-HD_NLSL and V5_Charentais_NLSL), either comprising the sgr-1 mutation or wild-type allele.

FIG. 13 shows a measurement of the firmness for different melon (V2_ItalianNet_NLSL, V4-HD_NLSL and V5_Charentais_NLSL) either comprising the sgr-1 mutation or wild-type allele.

### DETAILED DESCRIPTION

According to a first aspect, the present invention relates to a Cucumis melo plant, wherein said plant homozygously comprises in its genome a mutant allele of the staygreen (sgr) gene on chromosome 9, wherein said mutant allele of the sgr gene comprises at least one loss-of-function mutation in comparison to the sequence of a wild-type sgr allele (SEQ ID NO:1) and wherein said mutant allele of the sgr gene confers rind color stability to the fruits of said plant at maturity and/or during post-harvest, in comparison with an isogenic non-long shelf life (non-LSL) Cucumis melo plant which does not comprise said mutant allele. By homozygously comprising a mutant allele (loss-of-function) of the sgr gene, it is to be understood that a mutant allele of the sgr gene is present on every homologs of chromosome 9, but not necessarily the same mutant allele, provided all mutant alleles are indeed loss-of-function mutations.

In one embodiment, the non-LSL C. melo plant is a traditional C. melo plant. In such a case, the corresponding isogenic mutant plant is an ISL melon type or a LSL melon type. In one embodiment, the non-LSL C. melo plant is an ISL C. melo plant. In such a case, the corresponding isogenic mutant plant is a LSL melon type.

The melon plants of the invention are characterized by an homozygously inactivated sgr gene. The sgr gene has been mapped to chromosome 9 of the melon genome (NCBI GeneID 103482692). A sequence of a wild-type allele of the sgr gene is set forth in SEQ ID NO: 1. A coding sequence of a wild-type allele of the sgr gene is set forth in SEQ ID NO:3, and has been deposited in Genbank under accession XM_008438967 (update Jun. 7, 2016), wherein the coding sequence is positioned between nucleotides 415 and 1188. The translated sequence, i.e. the wild-type amino acid sequence of the SGR protein has been deposited in Genbank under accession XP_008437189.1 (update Jun. 7, 2016), as set forth in SEQ ID NO: 4.

In one embodiment, the mutant allele of the sgr gene is a loss-of-function allele, i.e. it comprises at least one loss-of-function mutation. The sequence of the mutant allele can differ from the wild-type sequence of the gene by at least one nucleotide substitution, insertion or deletion in said sequence. In particular, the mutation can be a single nucleotide polymorphism (SNP). The mutant allele of the sgr gene can also differ from the wild-type sequence of the sgr gene by the insertion or the deletion of one or more nucleic acid segments, including the deletion of the full gene. The mutation may induce one or more amino acid substitutions in the sequence of the SGR protein, and impair the function of the SGR protein.

In one embodiment, the loss-of-function mutation in the sgr gene is a null mutation. A null mutation prevents expression of an active SGR protein. Said mutation can be a nonsense mutation, causing a premature stop in the translation of the mRNA into a protein, resulting into the expression of a truncated form of the SGR protein. Alternatively, said mutation can be a framework mutation, causing a framework shift which results into the translation of an aberrant string of amino acids. Alternatively, said mutation can be a defective splicing mutation, causing errors in the splicing of the pre-mRNA into mature mRNA. Said mutation can be a splice site mutation, i.e. a mutation located in a splicing site of the gene, or it can be located in any splicing regulatory sequence, either in an intron or in an exon.

In the present invention, nonsense, framework or defective splicing mutations have the advantage that they generally result into the complete absence of expression of a functional protein, by way of contrast with a missense mutation (single amino acid substitution), for which the protein is most often expressed, and its activity may be partially retained.

The loss-of-function mutation may be located in any exon or intron of the sgr gene. In particular, the mutation is located in one of the first, second or third exons, or first, second or third introns.

According to one aspect, the mutation is a nucleotide substitution in the splicing site between the first intron and the second exon. In one embodiment, the mutation consists in the substitution of the guanine in last position of the first intron, by an alanine. This guanine is in position 584 of SEQ ID NO:1. This splicing site mutation, designated sgr-1, has been identified by the inventors in an EMS mutant plant and introgressed into different non-LSL and LSL genotypes. The sequence of the sgr-1 allele is set forth in SEQ ID NO:2.

Mutant alleles and corresponding markers can be identified by methods known in the art.

The mutation in the mutant sgr allele can be induced via methods such as mutagenesis or by means of genetic engineering. Mutagenesis methods and methods of genetic engineering are known in the art and are described below in more details.

Accordingly, the plants according to the invention may be obtained by different processes, and are not exclusively obtained by means of an essentially biological process.

Melon fruits according to the invention are characterized by an increased rind color stability at maturity and during post-harvest in comparison with an isogenic non-LSL fruit which does not comprises the mutant allele of the sgr gene, as defined herein. Stability of the rind color can be assessed by comparing the rind color of mutant and isogenic non-mutant melons at different time points, from maturity, preferably the day of the harvest, to postharvest, preferably between 7 days and 21 days post-harvest, in particular between 7 days and 14 days post-harvest, most particularly at 7 days or 14 days post-harvest. Preferably, stability of the rind color is assessed after 7 to 21 days in cold storage conditions at temperature comprised between 4° C. to 15° C. The same parameters are applicable to the measurement of any properties of the melons of the invention, or their isogenic non-mutant counterparts.

In some embodiments, the rind color of the melons is assessed by colorimetry either using a colorimeter like Konica Minolta CR400 or 2D image analysis from fruit pictures. Color measurements can be expressed in the CIELAB color space (also known as CIE L*a*b*). CIELAB color space is a color space defined by the International Commission on Illumination (CIE) in 1976. It expresses color as three values: L* for the lightness from black (0) to white (100), a* from green (−) to red (+), and b* from blue (−) to yellow (+). CIELAB was designed so that the same amount of numerical change in these values corresponds roughly to the same amount of visually perceived change. In this color space, a melon fruit visually perceived as greener has a lower a* value, whilst a melon visually perceived as yellower has a higher b* value.

In one embodiment, the melon fruit according to the invention is characterized by a lower a* value and/or a lower b* value at maturity and/or during post-harvest in comparison with an isogenic non-LSL melon fruit which do not comprise the mutant allele of the sgr gene. In one embodiment, the difference between the a* values and/or the b* values of melon fruits according to the invention and isogenic non-LSL melon fruits which do not comprise the mutant allele of the sgr gene is statistically significant. In one embodiment, the a* value and/or b* value of a melon fruit of the invention is inferior by at least 10%, preferably 20%, still preferably 30% to the a* value and/or b* value, respectively, of an isogenic non-LSL melon fruit which does not comprise the mutant allele of the sgr gene.

A color difference in the CIELAB color space can also be evaluated by the formula ΔE*=\sqrt{(L*₂−L*₁)²+(a*₂−a*₁)²+(b−b*₁)²}, wherein a color difference is reflected by a non-null ΔE*, as assessed by pairwise comparison statistical tools. In one embodiment, the ΔE* value of the rind color between a melon fruit according to the invention and an isogenic non-LSL melon fruit which do not comprise the mutant allele of the sgr gene is higher than 1, preferably higher 2, still preferably higher than 10, more preferably higher than 50.

In one embodiment, the rind color difference between melon fruits according to the invention and isogenic non-LSL melon fruits which do not comprise the mutant allele of the sgr gene is statistically significant.

Rind color differences with respect to non-mutant melons can also be assessed with the naked eye, for instance using color evaluation tools.

Melons according to the invention are also characterized by the fact that they retain several properties of non-LSL melons unchanged or substantially unchanged, such as their brix, their firmness, the rate of peduncle abscission and aroma, or their flesh color. These non-LSL like properties are particularly advantageous for the grower and the consumer, and are thus commercially valuable.

In one embodiment, the degree of brix of a fruit of a melon plant according to the invention at maturity and/or during post-harvest is substantially unchanged in comparison with the fruits of an non-LSL isogenic plant at the same stage of maturity and grown in the same environmental condition, wherein said isogenic plant does not comprise homozygously in its genome said mutant allele of the sgr gene.

In particular, the degree of brix of a fruit of a melon plant according to the invention is changed by less than 20%, preferably less than 10%, still preferably less than 5% in comparison with the fruit of an isogenic non-mutant plant.

The term “Degree brix” or “brix” indicates the soluble solid content of an aqueous solution inter alia of a juice, the vast majority of which being sugars. These are mostly estimated by a refractometer and measured as degrees Brix. The higher the degree, the more sugar content. The brix measurement is important to assess melon taste as fruits with low brix and therefore poor sugar content will not be appreciated by customers. Brix can be measured with a brixmeter, also known as a refractometer, as known by the skilled person.

Retaining the same brix, or substantially the same brix, as non-LSL isogenic melons not comprising the sgr mutation, is particularly advantageous, since the melons according to the invention cumulate the sweetness of non-LSL melons, in particular traditional melons with a longer shelf life. The melons of the invention thus avoid a typical drawback of LSL melons, in which the increased shelf life is generally associated with a lack of flavor.

In one embodiment, the firmness of the fruits of a melon plant according to the invention at maturity and/or during post-harvest is substantially unchanged in comparison to the fruits of an isogenic plant at the same stage of maturity and grown in the same environmental condition, wherein said isogenic plant does not comprise homozygously in its genome said mutant allele of the sgr gene.

In particular, the firmness of the fruits of a melon plant according to the invention is changed by less than 20%, preferably less than 10% in comparison with the fruits of said isogenic plant.

Firmness can be measured by a penetrometer, as known by the skilled person.

Non-LSL melons gradually lose their firmness at maturity, by an ethylene-dependent process. Melons of the invention show firmness properties similar or essentially similar to non-LSL melons, and thus tend to soften at maturity in a similar way as non-LSL melons.

In one embodiment, the degree of peduncle abscission of the fruits of a melon plant according to the invention at maturity and/or during post-harvest, is substantially unchanged in comparison with the fruits of an isogenic plant at the same stage of maturity and grown in the same environmental condition, wherein said isogenic plant does not comprise homozygously in its genome said mutant allele of the sgr gene.

In particular, the degree of peduncle abscission of the fruits of a melon plant according to the invention is changed by less than 20%, preferably less than 10% in comparison with fruits of said isogenic plant.

Peduncle abscission is a good indicator of maturity. At commercial maturity, in general non-LSL melon types form an abscission layer at the peduncle attachment whilst LSL melon types do not abscise. For this reason, LSL melons are also termed non-slip melon fruit, as they need to be cut from the vine, to be harvested. The presence of a peduncle abscission layer is thus particularly helpful for growers to determine when melons can be harvested. The melons of the invention have the advantage that they have a visible abscission layer is similar, or essentially similar, in terms of aspect and evolution, to isogenic non-mutant non-LSL plants.

The stage of peduncle abscission can be assessed visually on a scale from 1 to 9, wherein 1=full sipping and 9=not slipping.

In one embodiment, the cycle length of the fruits of a melon plant according to the invention at maturity is substantially unchanged in comparison with the fruits of a isogenic plant, grown in the same environmental conditions, wherein said isogenic plant does not comprise homozygously in its genome said mutant allele of the sgr gene.

In particular, the cycle length of a fruit of a plant according to the invention is changed by less than 20%, preferably less than 10%, still preferably less than 5% in comparison with the fruit of said isogenic plant. The cycle length corresponds to the period of time, e.g. in number of days, between the planting date and the harvest date. Maintaining a similar cycle length and therefore the same harvest windows as non-LSL types of melons is desirable since non-LSL melons become harvestable earlier than LSL melons, i.e. their cycle length is shorter compared to LSL melons, resulting in an improved yield.

The sgr mutation of the melon plants according to the invention can also have an effect on the leaves, and more particularly on the leaf color. In particular, the plants of the invention show reduced leaf yellowing and necrosis.

In one embodiment, the flesh color of the fruits of a melon plant according to the invention at maturity and/or during post-harvest is substantially unchanged in comparison to the fruits of an isogenic plant at the same stage of maturity and grown in the same environmental condition, wherein said isogenic plant does not comprise homozygously in its genome said mutant allele of the sgr gene. In particular, the a* and/or b* value of the flesh color of the fruits of a melon plant according to the invention is changed by less than 20%, preferably less than 10% in comparison with fruits of said isogenic plant. A flesh color difference may also be assessed in the CIELAB color space by the formula ΔE*. In one embodiment, the ΔE* value of the flesh color between a melon fruit according to the invention and an isogenic non-LSL melon fruit which does not comprise comprise homozygously in its genome the mutant allele of the sgr gene is lower than 50, preferably lower than 10, still preferably lower than 2, more preferably lower than 1.

In one embodiment, leaves of the melon plant according to the invention show reduced yellowing in comparison to an isogenic non-LSL plant, and wherein said isogenic plant does not comprise homozygously in its genome said mutant allele of the sgr gene.

Leaf color can be assessed with the naked eye or by colorimetry using a colorimeter. In particular, the CIElab color system can be used.

In one embodiment, the leaf color of a melon plant according to the invention is characterized by a lower a* value and/or a lower b* value in comparison with an isogenic non-LSL plant which do not comprise the mutant allele of the sgr gene.

Assessment of leaf color can thus be used as a surrogate for the identification of non-LSL plants displaying the phenotype of interest, namely rind color stability at maturity and/or during post-harvest.

The reduced leaf yellowing exhibited by the plants of the invention is also reflected as a resistance, and more specifically a partial resistance of the plants of the invention to yellowing diseases, such as CYSDV (Cucurbit Yellow Stunting Disorder Virus).

Accordingly, in some embodiments, a melon plant according to the invention is resistant to the CYSDV (Cucurbit Yellow Stunting Disorder Virus), wherein such resistance is provided by the allele mutant of the sgr gene. In particular, the resistance is a partial resistance.

CYSDV is a closterovirus transmitted in nature by the whitefly Bemisia tabaci. CYSDV induces interveinal chlorotic spots in mature leaves which may enlarge and eventually fuse together producing yellowing of the entire leaf except for the veins that remain green. Yellowing symptoms are accompanied by substantial reduction in fruit yield and quality and, therefore, the virus has a high economic importance.

The sgr mutation of the melon plants according to the invention can reduce the damages caused by CYSDV by hiding certain symptoms of the CYSDV on infected plants, in particular leaf yellowing. The resistance to CYSDV is advantageously determined by comparison to a susceptible (commercial) line.

In one embodiment, the C. melo plant according to the invention is a plant from an inbred C. melo line.

In a preferred embodiment, the C. melo plant according to the invention is an F1 hybrid C. melo plant.

The invention also relates to a group of C. melo plants according to the invention, wherein said population comprises at least 5 plants, in particular at least 10 plants, more particularly at least 20 plants, even more particularly at least 50 or 100, or more particularly at least 1000 plants.

The invention is also directed to further aspects, as detailed below. All the embodiments detailed in the preceding section in connection with the first aspect of the invention are also embodiments according to these further aspects of the invention.

According to a second aspect, the present invention is directed to a cell of a C. melo plant according to the invention, wherein said cell comprises in its genome a mutant allele of the staygreen (sgr) gene on chromosome 9, wherein said mutant allele of the sgr gene comprises at least one loss-of-function mutation in comparison to the sequence of a wild-type sgr allele (SEQ ID NO:1).

A plant cell of the invention may have the capacity to be regenerated into a whole plant.

Alternatively, the invention is also directed to plant cells which are not regenerable, and thus are not capable of giving rise to a whole plant.

According to an embodiment, the cell is derived from an embryo, protoplast, meristematic cell, callus, pollen, leaf, anther, stem, petiole, root, root tip, fruit, seed, flower, cotyledon, and/or hypocotyl.

In one aspect, the invention is directed to a plant part of a C. melo plant of the invention. The invention also relates to a plant part of C. melo plant comprising at least one cell according to the invention.

According to one embodiment, the plant part is an embryo, protoplast, meristematic cell, callus, pollen, leaves, anther, stem, petiole, root, root tip, fruit, seed, flower, cotyledon, and/or hypocotyl. In one embodiment, the plant part is a fruit of a C. melo plant according to the invention.

Another aspect of the invention relates a C. melo seed, which can be grown into a C. melo plant according to the invention. Such seed are thus ‘seed of a plant of the invention’, i.e. seed giving rise to a plant of the invention. The invention is also concerned with a seed from a plant of the invention, i.e. obtained from such a plant after selfing or crossing, provided however that the plant obtained from said seed homozygously comprise a loss-of-function mutant allele of the sgr gene conferring rind color stability to the fruits of said plant at maturity and/or during post-harvest, in comparison with an isogenic non-LSL C. melo plant which does not comprise said mutant allele.

The invention also relates to a group of C. melo seeds according to the invention, wherein said population comprises at least 2 seeds, especially at least 10 seeds, particularly at least 100 seeds, even more particularly at least 1000 seeds.

Another aspect of the invention in vitro cell or tissue culture of regenerable cells of the C. melo plant according to the invention. Preferably, the regenerable cells are derived from an embryo, protoplast, meristematic cell, callus, pollen, leaf, anther, stem, petiole, root, root tip, seed, flower, cotyledon, and/or hypocotyl. Said regenerable cells comprise in their genome a loss-of-function mutant allele of the sgr gene as described above.

The tissue culture will preferably be capable of regenerating plants having the physiological and morphological characteristics of the foregoing C. melo plant, and of regenerating plants having substantially the same genotype as the foregoing C. melo plant. The present invention also provides C. melo plants regenerated from the tissue cultures of the invention.

The invention also provides a protoplast of the plant defined above, or from the tissue culture defined above, said protoplast comprising in its genome a loss-of-function mutant allele of the sgr gene as described here above.

According to another aspect, the present invention is also directed to the use of a C. melo plant as detailed according this invention, as a breeding partner in a breeding program for obtaining C. melo plants with increased shelf life, in particular with increased rind color stability at maturity and/or during post-harvest. Indeed, such a C. melo plant according to the first aspect harbors in its genome a loss-of-function allele of the sgr gene, as defined here above, conferring rind color stability at maturity and/or during post-harvest. By crossing this plant with plants not comprising the mutation, it is thus possible to transfer this allele, conferring the desired phenotype, to the progeny. A plant according to the invention can thus be used as a breeding partner for introgressing the mutant allele conferring the desired phenotype into a C. melo plant or germplasm.

In such a breeding program, the selection of the progeny displaying the desired phenotype, or bearing sequences linked to the desired phenotype, can advantageously be carried out on the basis of the allele as disclosed here above, and corresponding markers.

The invention is also directed to the use of said plants in a program aiming at identifying, sequencing and/or cloning the genetic sequences conferring the desired phenotype.

According to another aspect, the invention also concerns methods for the production of C. melo plants having an increased shelf life, especially commercial plants. A method or process for the production of a plant having these features comprises the following steps:
(a1) crossing a C. melo plant according to present invention, homozygously comprising a mutant allele of the sgr gene, wherein the sequence of said mutant allele of the sgr gene comprises at least one loss-of-function mutation in comparison to the sequence of a wild-type sgr allele (SEQ ID NO:1), with a second C. melo plant which does not homozygously comprise said mutant allele, thus generating a F1 population,
(a2) advancing the F1 population to create F2 population,
(b) selecting one plant homozygously comprising said mutant allele in the progeny thus obtained;
(c) optionally self-pollinating one or several times the plant obtained at step b);
(d) optionally backcrossing the plant selected in step b) or c) with a C. melo plant which does not homozygously comprise said mutant allele, and
(e) selecting a plant homozygously comprising said mutant allele, wherein said plant produces fruits with an increased shelf life,
(f) optionally crossing the selected plant with a different C. melo plant homozygously comprising said mutant sgr allele, thereby producing a hybrid C. melo plant homozygously comprising said mutant sgr allele.

The plant selected at step e) or produced at step (f) is preferably a commercial variety, cultivar or type of melon. In some embodiments, the selected plant is from one of the types Charentais, Italian netted, Western Shipper, Eastern Shipper, Galia, Ananas and Honey Dew.

Preferably, steps c) and/or d) are repeated at least twice and preferably three times, not necessarily with the same C. melo plant which does not homozygously comprise said mutant allele. Said C. melo plant which does not homozygously comprise said mutant allele is preferably a breeding line.

The self-pollination and backcrossing steps may be carried out in any order and can be intercalated, for example a backcross can be carried out before and after one or several self-pollinations, and self-pollinations can be envisaged before and after one or several backcrosses.

In some embodiments, such a method is advantageously carried out by using nucleic acid markers for one or more of the selections carried out at steps b) or e) for selecting plants homozygously comprising said mutant allele of the sgr gene.

The selection carried out at steps b) or e) can be made using any type of genetic marker, in particular restriction fragment length polymorphisms (RFLPs), amplified fragment length polymorphisms (AFLPs), simple sequence repeats (SSRs), simple sequence length polymorphisms (SSLPs), single nucleotide polymorphisms (SNPs), insertion/deletion polymorphisms (Indels), variable number tandem repeats (VNTRs), and random amplified polymorphic DNA (RAPD), isozymes, and other markers known to those skilled in the art.

The method used for marker and allele detection can be based on any technique allowing the distinction between two different alleles of a marker, on a specific chromosome. Detection of a polymorphism can be made by electrophoretic techniques including a single strand conformational polymorphism (Orita, et al. (1989) Genomics, 8(2), 271-278), denaturing gradient gel electrophoresis (Myers (1985) EPO 0273085), or cleavage fragment length polymorphisms (Life Technologies, Inc., Gaithersburg, Md.), but the widespread availability of DNA sequencing often makes it easier to simply sequence amplified products directly. Once the polymorphic sequence difference is known, rapid assays for the detection of a polymorphism can be designed for progeny testing, generally involving some version of PCR amplification of specific alleles (PASA; Sommer, et al. (1992) Biotechniques 12(1), 82-87), or PCR amplification of multiple specific alleles (PAMSA; Dutton and Sommer (1991) Biotechniques, 11(6), 700-7002). In particular examples, PCR detection and quantification is carried out using two labeled fluorogenic oligonucleotide forward primers and an unlabeled common reverse primer, for example, KASPar™ (KBiosciences). Detection of a polymorphism can also be made by electrophoretic techniques including a single strand conformational polymorphism (Orita, et al. (1989) Genomics, 8(2), 271-278), denaturing gradient gel electrophoresis (Myers (1985) EPO 0273085), or cleavage fragment length polymorphisms (Life Technologies, Inc., Gaithersburg, Md.). The widespread availability of DNA sequencing often also enables to sequence amplified products directly.

The present invention also concerns a C. melo plant obtained or obtainable by the methods described herein. Such a plant is indeed a C. melo plant having the characteristics described in the first aspect of the invention.

The plant is preferably a commercial variety, cultivar or type of melon. The plant is preferably a F1 hybrid melon plant. In some embodiments, the plant is one of the following types: Charentais, Italian netted, Western Shipper, Eastern Shipper, Galia, Ananas and Honey Dew.

Also provided are methods for producing C. melo plants seeds. In some embodiments, the methods comprise crossing the C. melo plant according to the invention with itself or with another C. melo plant, and harvesting the resultant seeds.

In addition to introgression of the mutant allele of the sgr gene, as detailed in the methods of the invention, said sequence can also be introduced into C. melo background by genetic engineering in order to obtain a commercial C. melo plant having the advantageous features of the invention, in particular an increased shelf life. The identification and cloning of the introgressed mutant allele conferring the desired phenotype are routine for the skilled person.

It is noted that the seeds or plants of the invention may be obtained by different processes, in particular technical processes such as mutagenesis, e.g. chemical mutagenesis or UV mutagenesis, or genetic engineering such as guided recombination or genome editing, and are not exclusively obtained by means of an essentially biological process.

In one embodiment, the invention is directed to a method of producing a C. melo plant producing fruits or susceptible to produce fruits with an increased shelf life, comprising the introduction of a loss-of-function mutation in the sgr gene on chromosome 9 in the genome of a non-LSL C. melo plant, wherein said mutation is introduced by mutagenesis or genome editing, in particular by a technique selected from ethyl methanesulfonate (EMS) mutagenesis, oligonucleotide directed mutagenesis (ODM), Zinc finger nuclease (ZFN) technology, Transcription Activator-Like Effector Nucleases (TALENs) the CRISPR/Cas system, engineered meganuclease, re-engineered homing endonucleases and DNA guided genome editing. Preferably, a loss-of-function mutation is introduced in all the copies of the sgr gene, present on the chromosomes 9.

In particular, one embodiment of the invention relates to a method for obtaining a C. melo plant producing fruits or susceptible to produce fruits with an increased shelf life, or a seed thereof, said method comprising:
a) treating MO seeds of a C. melo plant to be modified, preferably a non-LSL C. melo plant, with a mutagenic agent to obtain M1 seeds;
b) growing plants from the thus obtained M1 seeds to obtain M1 plants;
c) producing M2 seeds by self-fertilisation of M1 plants; and
d) optionally repeating step b) and c) n times to obtain M2+n seeds.

In this method, the M1 seeds of step a) can be obtained via chemical mutagenesis such as EMS mutagenesis, or by any other chemical mutagenic agents include but are not limited to, diethyl sufate (des), ethyleneimine (ei), propane sultone, N-methyl-N-nitrosourethane (mnu), N-nitroso-N-methylurea (NMU), N-ethyl-N-nitrosourea(enu), and sodium azide. Alternatively, the mutations are induced by means of irradiation, which is for example selected from x-rays, fast neutrons, UV radiation.

In another embodiment of the invention, the mutations are induced by means of genetic engineering. Such mutations also include the integration of sequences conferring the phenotype of the mutant plants according to the invention, in particular rind color stability as well as the substitution of residing sequences by alternative sequences conferring the phenotype of the mutant plants according to the invention, in particular rind color stability.

The genetic engineering means which can be used include the use of all such techniques called New Breeding Techniques which are various new technologies developed and/or used to create new characteristics in plants through genetic variation, the aim being targeted mutagenesis, targeted introduction of new genes or gene silencing (RdDM). Example of such new breeding techniques are targeted sequence changes facilitated thru the use of Zinc finger nuclease (ZFN) technology (ZFN-1, ZFN-2 and ZFN-3, see U.S. Pat. No. 9,145,565, incorporated by reference in its entirety), Oligonucleotide directed mutagenesis (ODM), Cisgenesis and intragenesis, RNA-dependent DNA methylation (RdDM, which does not necessarily change nucleotide sequence but can change the biological activity of the sequence), Grafting (on GM rootstock), Reverse breeding, Agro-infiltration (agro-infiltration “sensu stricto”, agro-inoculation, floral dip), Transcription Activator-Like Effector Nucleases (TALENs, see U.S. Pat. Nos. 8,586,363 and 9,181,535, incorporated by reference in their entireties), the CRISPR/Cas system (see U.S. Pat. Nos. 8,697,359; 8,771,945; 8,795,965; 8,865,406; 8,871,445; 8,889,356; 8,895,308; 8,906,616; 8,932,814; 8,945,839; 8,993,233; and 8,999,641, which are all hereby incorporated by reference), engineered meganuclease re-engineered homing endonucleases, DNA guided genome editing (Gao et al., Nature Biotechnology (2016), doi: 10.1038/nbt.3547, incorporated by reference in its entirety), and Synthetic 5 genomics). A major part of today's targeted genome editing, another designation for New Breeding Techniques, is the applications to induce a DNA double strand break (DSB) at a selected location in the genome where the modification is intended. Directed repair of the DSB allows for targeted genome editing. Such applications can be utilized to generate mutations (e.g., targeted mutations or precise native gene editing) as well as precise insertion of genes (e.g., cisgenes, intragenes, or transgenes). The applications leading to mutations are often identified as site-directed nuclease (SDN) technology, such as SDN1, SDN2 and SDN3. For SDN1, the outcome is a targeted, non-specific genetic deletion mutation: the position of the DNA DSB is precisely selected, but the DNA repair by the host cell is random and results in small nucleotide deletions, additions or substitutions. For SDN2, a SDN is used to generate a targeted DSB and a DNA repair template (a short DNA sequence identical to the targeted DSB DNA sequence except for one or a few nucleotide changes) is used to repair the DSB: this results in a targeted and predetermined point mutation in the desired gene of interest. As to the SDN3, the SDN is used along with a DNA repair template that contains new DNA sequence (e.g. gene). The outcome of the technology would be the integration of that DNA sequence into the plant genome. The most likely application illustrating the use of SDN3 would be the insertion of cisgenic, intragenic, or transgenic expression cassettes at a selected genome location. A complete description of each of these techniques can be found in the report made by the Joint Research Center (JRC) Institute for Prospective Technological Studies of the European Commission in 2011 and titled “New plant breeding techniques—State-of-the-art and prospects for commercial development”, which is incorporated by reference in its entirety.

DNA-editing technologies are successfully used in melons for inactivating targeted genes, at specific positions (Hooghvorst, et al. “Efficient knockout of phytoene desaturase gene using CRISPR/Cas9 in melon.” Scientific reports 9.1 (2019): 1-7).

The present invention also provides methods for identifying, detecting and/or selecting C. melo plants producing fruits or susceptible to produce fruits with an increased shelf life, said method comprising the detection of a mutant allele of the sgr gene on chromosome 9 in the genome of said plants, wherein said mutant allele comprises at least one loss-of-function mutation in comparison to the sequence of a wild-type sgr allele (SEQ ID NO:1).

In one embodiment, said loss-of-function allele is selected from a nonsense mutation, an indel mutation, a framework mutation or a defective splicing mutation, in particular a splicing site mutation.

In one embodiment, said method comprises the detection of a substitution of a guanine in position 584 of SEQ ID NO:1, i.e. allele sgr-1, the sequence of which is set forth in SEQ ID NO:2.

In some embodiments, detection of the mutant allele of the sgr gene is performed by amplification, e.g. by PCR, using, for each marker, one forward primer which can be used for amplifying the resistant allele, one forward primer which can be used for amplifying the susceptible allele and one common reverse primer, for example using the KASPar™ (KBiosciences) technology. In particular, the primers for amplifying each of said markers may have the sequences as described in Table 1.

In a preferred embodiment, the amplification is performed using a two-step touchdown method in which the elongation and annealing steps are incorporated into a single step. The temperature used for the annealing stage determines the specificity of the reaction and hence the ability of the primers to anneal to the DNA template. A touchdown PCR involves a first step of Taq polymerase activation, followed by a second step called the touchdown step that involves a high annealing temperature and incrementally decreasing the annealing temperature in each PCR cycle, and a third step of DNA amplification. The higher annealing temperatures in the early cycles of a touchdown ensure that only very specific base pairing will occur between the DNA and the primer, hence the first sequence to be amplified is most likely to be the sequence of interest. The annealing temperature is gradually decreased to increase the efficiency of the reaction. The regions that were originally amplified during the highly specific early touchdown cycles will be further amplified and outcompete any non-specific amplification that may occur at the lower temperatures.

In another embodiment, the amplification of SNP markers is performed as recommended in the KASPar assay and illustrated in the examples (see example 1).

According to a further aspect, the present invention also provides one or more molecular marker(s) for identifying a C. melo plant producing fruits or susceptible to produce fruits with an increased shelf life, wherein said molecular marker detects a loss-of-function mutation in the sgr gene on chromosome 9.

Further provided is the use of one or more molecular markers, for detecting a C. melo plant producing fruits or susceptible to produce fruits with an increased shelf life, wherein said molecular marker(s) detect(s) a loss-of-function mutation in the sgr gene on chromosome 9 in the sgr gene on chromosome 9.

According to these aspects of the invention, the molecular marker may be located in the sgr gene or in a chromosomal region in genetic linkage with the sgr gene. In one embodiment, said molecular marker(s) identifies a substitution of guanine at position 584 of SEQ ID NO:1 by an alanine. In one embodiment, said molecular marker is located within the sequence set forth in SEQ ID NO:5.

The invention is also directed to a method for identifying a molecular marker suitable for detection of a C. melo plant producing fruits or susceptible to produce fruits with an increased shelf life, comprising:
(a) identifying a molecular marker in the sgr gene or in a chromosomal region in genetic linkage with the sgr gene,
(b) determining whether said molecular marker is associated with or linked with a phenotype of increased shelf life, in particular an increased rind color stability at maturity and/or during post-harvest, of the melon fruits.

In a further aspect, the invention relates to method for the production of C. melo plantlets or plants producing fruits or susceptible to produce fruits with an increased shelf life, which method comprises:
i. culturing in vitro an isolated cell or tissue of the C. melo plant according to the invention to produce C. melo micro-plantlets, and
ii. optionally further subjecting the C. melo micro-plantlets to an in vivo culture phase to develop into C. melo plants producing fruits or susceptible to produce fruits with an increased shelf life.

The isolated cell or tissue used to produce a micro-plantlet is an explant obtained under sterile conditions from a C. melo parent plant of the invention to be propagated. The explant comprises or consists, for instance, of a cotyledon, hypocotyl, stem tissue, leaf, embryo, meristem, node bud, shoot apice, or protoplast. The explant can be surface sterilized before being placed on a culture medium for micropropagation.

Conditions and culture media that can be suitably used in plant micropropagation are well known to those skilled in the art of plant cultivation and are described, for example, in “Plant Propagation by Tissue Culture, Handbook and Directory of Commercial Laboratories, eds. Edwin F George and Paul D Sherrington, Exegetics Ltd, 1984”.

Micropropagation typically involves:
1. axillary shoot production : axillary shoot proliferation is induced by adding cytokinin to the shoot culture medium, to produce shoots preferably with minimum callus formation;
ii. adventitious shoot production: addition of auxin to the medium induces root formation, in order to produce plantlets that are able to be transferred into the soil. Alternatively, root formation can be induced directly into the soil.

Plantlets can be further subjected to an in vivo culture phase, by culture into the soil under lab conditions, and then progressive adaptation to natural climate, to develop into a C. melo plant according to the invention.

The reduced leaf yellowing displayed by melons of the invention permits to reduce the yield losses caused by leaf yellowing in various physiological or pathological states, e.g. senescence or the presence of a yellowing disease such as a CYSDV infection. The invention is thus also directed to a method for improving the yield of C. melo plants or for increasing the number of harvestable melon plants or fruits, comprising growing C. melo plants according to the invention, homozygously comprising a mutant allele of the sgr gene on chromosome 9, wherein said mutant allele comprises at least one loss-of-function mutation and confers reduced leaf yellowing. In one embodiment, the C. melo plants according to the invention are grown in an environment infected by CYSDV.

Preferably, the method comprises a first step of choosing or selecting a C. melo plant comprising said mutant allele. The method can also be defined as a method of increasing the productivity of a melon field, tunnel or glasshouse, or as a method of reducing the intensity or number of chemical or fungicide applications in the production of melons.

The invention is also directed to a method for reducing the loss on C. melo production, comprising growing a C. melo plant as defined above. In particular, the C. melo plant is grown in conditions of CYSDV infection.

In another embodiment, the invention is directed to a method for protecting a melon field, tunnel or glasshouse, or any other type of plantation, from a yellowing disease, such as CYSDV infection, or of at least limiting the level of infection or limiting the spread of the disease. Such a method preferably comprises the step of growing a yellowing disease resistant plant of the invention, i.e. a plant comprising on chromosome 9 a mutant allele of the sgr gene, wherein said mutant allele comprises at least one loss-of-function mutation.

The invention also concerns the use of a C. melo plant resistant, in particular partially resistant, to a yellowing disease such as CYSDV, according to the invention, in a field, tunnel or glasshouse, or other plantation.

The present invention is also directed to a method for improving the yield of C. melo plants in an environment infested by CYSDV comprising:
(a) identifying C. melo plants resistant to CYSDV, wherein said plants homozygously comprise a mutant allele of the sgr gene on chromosome 9, said mutant allele comprising at least one loss-of-function mutation, and
(b) growing said tolerant C. melo plants in said infested environment.

By this method, the yield of the C. melo plants is increased, inter alia more marketable melons can be harvested, or more commercial melons are produced, or more seeds are obtained.

The invention further relates to a method for improving the shelf life of melon fruit, the marketability of melon fruit and/or the yield of melon production, wherein said method comprises growing C. melo plants according to the invention and harvesting fruits set by said plants. Thanks to their increased shelf life, melons according to the invention can be harvested at reduced frequency than existing non-LSL melons, in particular from 2 to 4 per weeks. Accordingly, the invention also relates to a method for increasing melon harvest flexibility, wherein said method comprises growing C. melo plants according to the invention and harvesting fruits set by said plants.

In one embodiment, of these methods, said fruits are stored at least 7 days, preferably between 7 to 21 days after harvest.

In still a further aspect, the invention also relates to a method of producing melon fruits comprising:
(a) growing a C. melo plant of the invention, as defined previously;
(b) allowing said plant to set fruit; and
(c) harvesting fruit of said plant, preferably at maturity and/or before maturity.

All the preferred embodiments regarding the C. melo plant are already disclosed in the context of the previous aspects of the invention.

The method may advantageously comprise a further step of processing said C. melo plant into a processed food.

In another aspect, the invention relates to the use of a C. melo plant according to the invention or a fruit thereof in the fresh cut market or for food processing.

Throughout the instant application, the term “comprising” is to be interpreted as encompassing all specifically mentioned features as well optional, additional, unspecified ones. As used herein, the use of the term “comprising” also discloses the embodiment wherein no features other than the specifically mentioned features are present (i.e. “consisting of”).

### EXAMPLES

### Example 1: Generation and Identification of a Mutant Melon by EMS Mutagenesis

Mature seeds from a climacteric Charentais cultivar were mutagenized by immersion in 1% to 3% ethyl-methanesulfonate (EMS) for 16 h, followed by washing with 0.1 M Na₂SO₃. The seeds were then rinsed and sown in soil. M2 seeds were collected from the M1 plants. Genomic DNA was extracted from the M2 plants, and a SNP on the sgr gene on chromosome 9 was identified, with a G->A substitution in the splicing site, at the end of the first intron. This mutant allele is designated sgr-1. Its sequence is set forth in SEQ ID NO:2.

The sgr-1 mutation can be identified using using the KASPar™ (KBiosciences) assay, with two labeled fluorogenic oligonucleotide forward primers and an unlabeled common reverse primer (Table 1).

TABLE 1
PCR primers for detection of sgr-1

| | | | | Sgr-1 |
|---|---|---|---|---|
| Forward primer (wild- | Forward primer (sgr- | Common reverse | wild-type | mutant |
| type allele)/FAM | 1 mutant allele)/VIC | primer | allele | allele |
| GAAGGTGACCAAGTTCA | GAAGGTCGGAGTC | GAACTTTCGAAAGT | G | A |
| TGCTGGCCCAAACAATC | AACGGATTCTGGC | GGGTTTTGTTTTGA | | |
| TCGCCATC (SEQ ID | CCAAACAATCTCG | TT (SEQ ID NO: 8) | | |
| NO: 6) | CCATT (SEQ ID | | | |
| | NO: 7) | | | |

### Example 2: Introgression of the sgr-1 Mutation

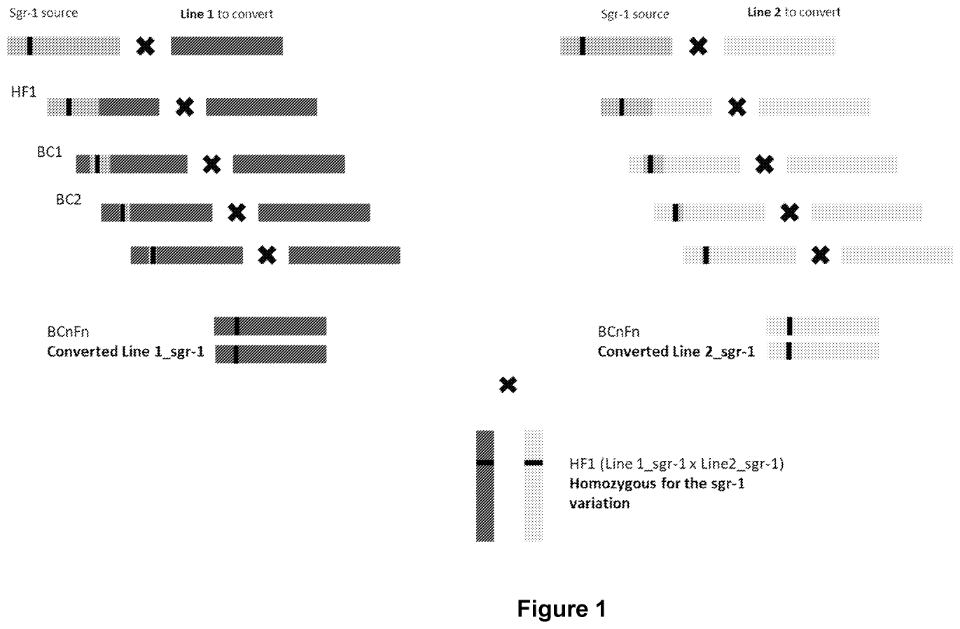

The sgr-1 mutation was introgressed in different elite genotypes. The sgr-1 mutation from the EMS population being recessive, its effect is only present when the variation is at an homozygous state. In order to produce HF1 hybrids exhibiting this effect, various parental lines were converted. After a first cross between the parental line and the sgr-1 source, the sgr-1 mutation was backcrossed several times in the parent line (FIG. 1). The two resulting converted lines were crossed together, thus generating HF1 hybrids homozygous for the sgr-1 mutation, which are near isogenic lines (NILs) with respect to HF1 which lack the sgr-1 mutation.

Several genotypes were converted with the sgr-1 mutation, including the following orange-flesh typologies: Charentais, Italian netted, Western Shipper, Eastern Shipper and the following white and green flesh typologies: Yellow Canary, Piel de Sapo, Galia and Honey Dew.

### Example 3: Effect of the sgr-1 Mutation on Leaves

In order to assess the effect of the sgr-1 mutation on leaf yellowing and necrosis, the inventors evaluated the leaf color of different melon genotypes, with or without the sgr-1 mutation.

Leaf color is evaluated at different times during the plant growing usually at an early stage before fruit setting (date1), during the fruits maturation (date2) and at a late stage during or just after fruit harvest (date3). Several plants are evaluated per genotype (between 5 and 10 plants) and leaf color can be assessed by eye and using a colorimeter. For example, Konica Minolta CR400 colorimeter can be used to measure leaf color. For a given date, 2 measures are realized on a plant before doing the average to get the average plant value for the three coordinates (L*, a* and b*) of the CIELAB color space. Measured leaves are chosen to be representative of the plant (not too young or too old). Then, average L*, a* and b* can be calculated at the genotype level for a given date using the mean of all the plant values.

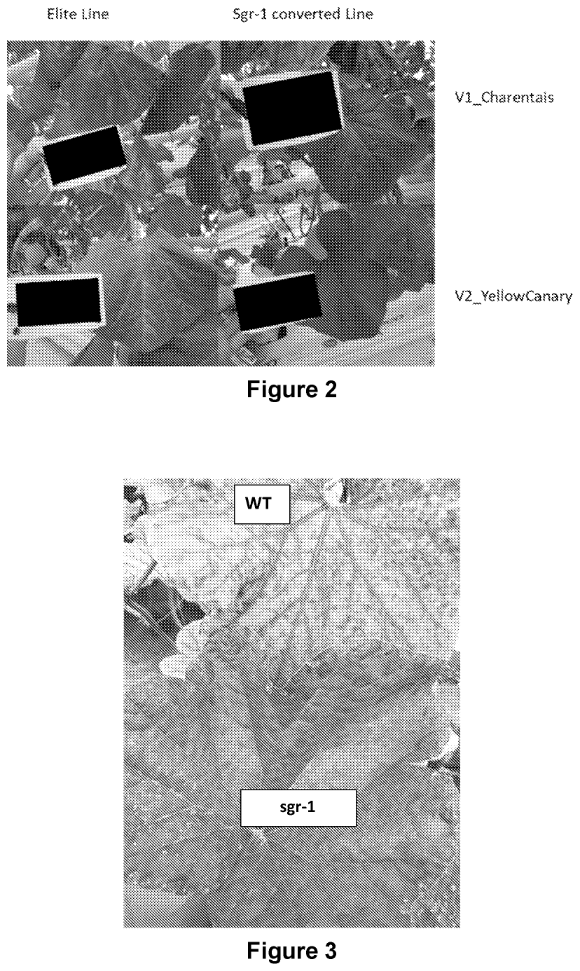

A significant effect on the leaf color was visibly observed on V1_Charentais and V2_Yellow Canary lines (FIG. 2).

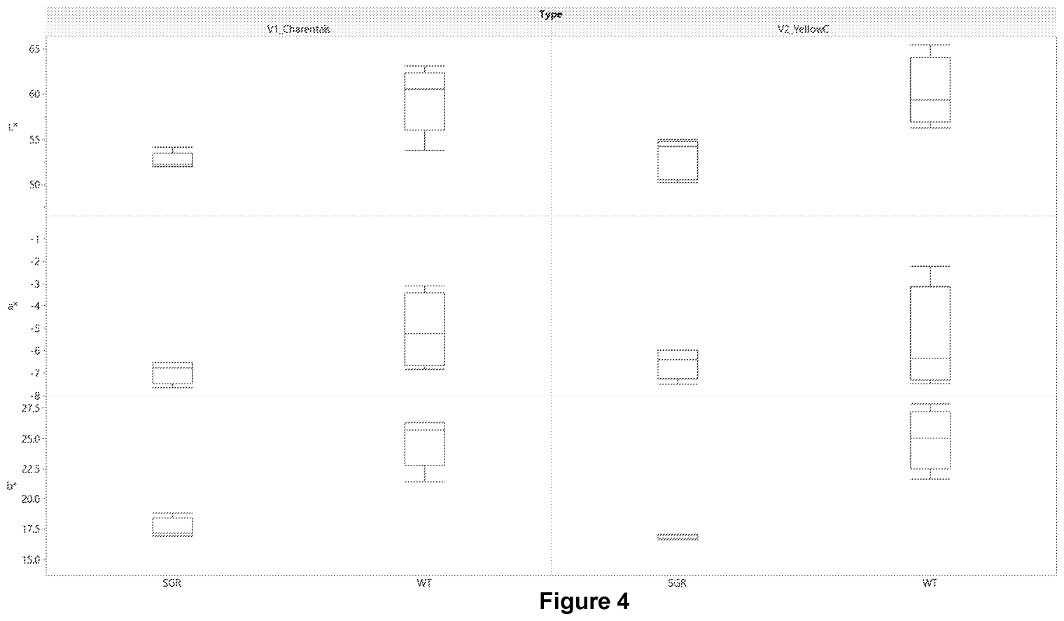

In order to evaluate more precisely the color evolution and difference, leaf color measurements were made using a colorimeter. The lower L*, a* and b* values which are observed for sgr-1 genotypes reflect darker and greener leaves color. Moreover, the reduced dispersion of the sgr-1 data shows the higher stability of the leave color and express a reduced leaf yellowing with the sgr-1 mutation in comparison with the initial genotype (FIG. 4).

Significant effect is at least recorded on one of the three coordinates (L*, a*, b*) when means between sgr-1 converted lines and original lines are compared using ANOVA test (Table 2). We can definitely conclude on a significant effect of sgr-1 variation on the leaf color especially during the fruit maturation stages and late stages.

TABLE 2
sgr-1 effect on leaf color

| | Sgr-1 Effect | Pvalue |
|---|---|---|
| V1_Charentais | L* | 0.0036** |
| | a* | 0.0409* |
| | b* | <0.0001** |
| V2_YellowC | L* | 0.0102* |
| | a* | 0.3949 |
| | b* | 0.0002** |

Calculation of the colorimetric difference, ΔE* using the formula below supports this conclusion.

ΔE*=\sqrt{(L*₂−L*₁)²+(a*₂−a*₁)²+(b*₂−b*₁)²}

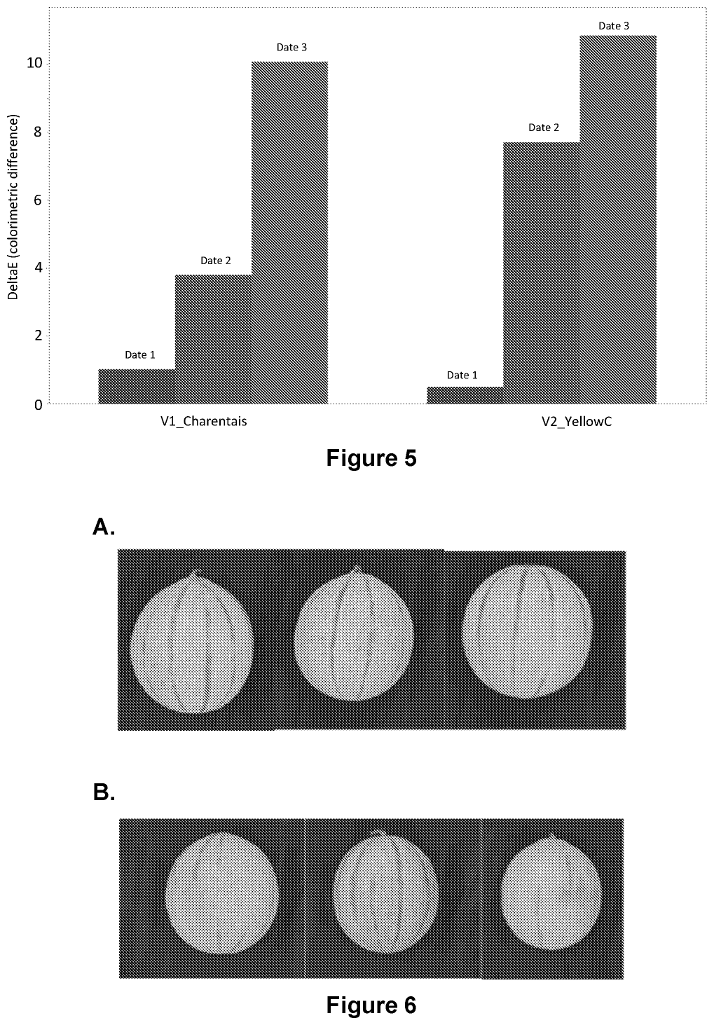

ΔE* is low at the early stage meaning that leaf colors are quite similar between the sgr-1 and the corresponding wild-type (WT) lines. Then, ΔE* is increasing with the time, it shows the evolution of the color difference which is more visible at the later plant stage (FIG. 5).

In addition of the sgr-1 effect on leaves visible in regular conditions, a strong sgr effect was also visible under CYSDV pressure (natural infection in an area seriously affected by the virus). The virus is still present on leaves of plants carrying sgr-1 mutant allele, but the visible symptoms are hidden and the plants carrying sgr-1 mutant allele present less yellowing than the plants with the wild-type allele counterpart. The sgr-1 mutation provides an interesting partial resistance to yellowing symptoms of CYSDV (FIG. 3).

### Example 4: Effect of the sgr-1 Mutation on the Rind Color

During the conversion of the lines, the inventors observed an effect of the sgr-1 mutation on the rind color, according to the melon type. Fruits tend to stay greener. The inventors assessed this effect visually and by colorimetry, both at the harvest day and 7 days after storage. Observations and measurements were conducted on different melon genotypes and more specifically on orange-flesh material on the 3 following varieties: V1_Charentais_LSL, V2_ItalianNet_NLSL, V3_ItalianNet_NLSL

On orange-flesh material, a color difference was clearly observable between V2_ItalianNet_NLSL WT version and sgr-1 converted variety at harvest. V2_ItalianNetted variety is non-LSL and turn yellow at maturity. The rind of the sgr-1 version stays however green.

After 7 days of storage, wild-type (WT) V2_ItalianNet_NLSL fruits turn more and more yellow orange whereas V2_ItalianNet_NLSL sgr-1 fruits don't evolve externally and keep their green rind color (FIG. 6). The color version of FIG. 6 makes apparent the effect of sgr-1 on the rind color. The color version of all Figures of the present application, including FIG. 6, is filed with the present application and available on request.

Observations were also made on V1_Charentais_LSL, an LSL genotype which does not turn yellow at maturity. At harvest, no color difference could be observed between the sgr-1 and the WT version of V1_Charentais_LSL . This shows that the sgr-1 genotype does not impact, or only to a minor extent, the rind color of LSL genotypes of melon.

In addition, recessive effect of the sgr variation was also confirmed on the rind color. No differences were observed on the WT version of V3_ItalianNet_NLSL and the heterozygote version for sgr-1.

Even if the sgr-1 effect is quite visible, colorimetric data can also be used to demonstrate it. Because of the netting presence on the fruit rind surface, colorimeter tool was not appropriate for such measures. Instead, image analysis can be used to extract the rind color and access to the L*, a* and b* values.

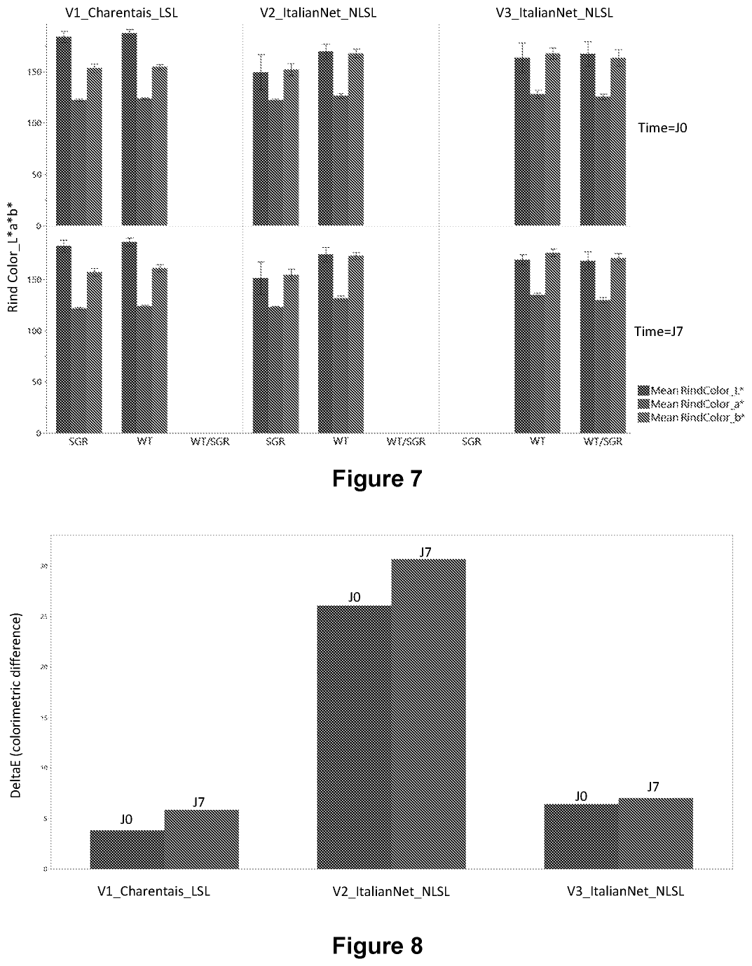

A significant statistical effect was observed for the sgr-1 variation on the Variety 2, Non-LSL genotype, in comparison of the WT version for the L* and b* values (FIG. 7). Higher L*a*b* values reflect a yellower color of the fruit rind of the WT version in comparison to the sgr-1 version, which is greener. No differences are observed between the sgr-1 and the WT versions of the Variety 1 which is LSL. Also, no significant differences were recorded between the heterozygous sgr-1 version and the WT version of the Non LSL Variety 3. The mutation needs to be at the homozygous state and in a non LSL background to reveal its effect on the rind color.

Colorimetric differences can be calculated thanks to the ΔE* formula between the sgr-1 converted version and the WT corresponding line. The effect of the sgr-1 variation on the Variety 2 is clear at J0 (day of harvest) and increases slightly at J7 (7 days of storage) with a ΔE* higher than 10 (26.1 and 30.7 respectively) whereas the ΔE* for the other varieties is lower than 10 (FIG. 8).

### Example 5: Effect of the sgr-1 Mutation on the Flesh Color

The inventors further assessed the effect of the sgr-1 mutation on the flesh color of the melons to control potential impact on this fruit quality trait.

The flesh color of different genotypes of melons was visually observed and measured by colorimetry, at postharvest stage, after 7 days of storage in cold room at 14° C. Two categories of melons were evaluated: orange-flesh material with the varieties V1_Charentais_LSL, V2_ItalianNet_NLSL, and white-flesh material with the variety V6_YellowC_LSL with their respective versions, the wild-type (WT) and the sgr-1 version.

10 fruits per genotype were evaluated for their flesh color. The measures are done on the equatorial slice of the fruit using a colorimeter. 2 diametrically opposed measurements are realised per fruit, then, the mean is calculated to get the color at the fruit level.

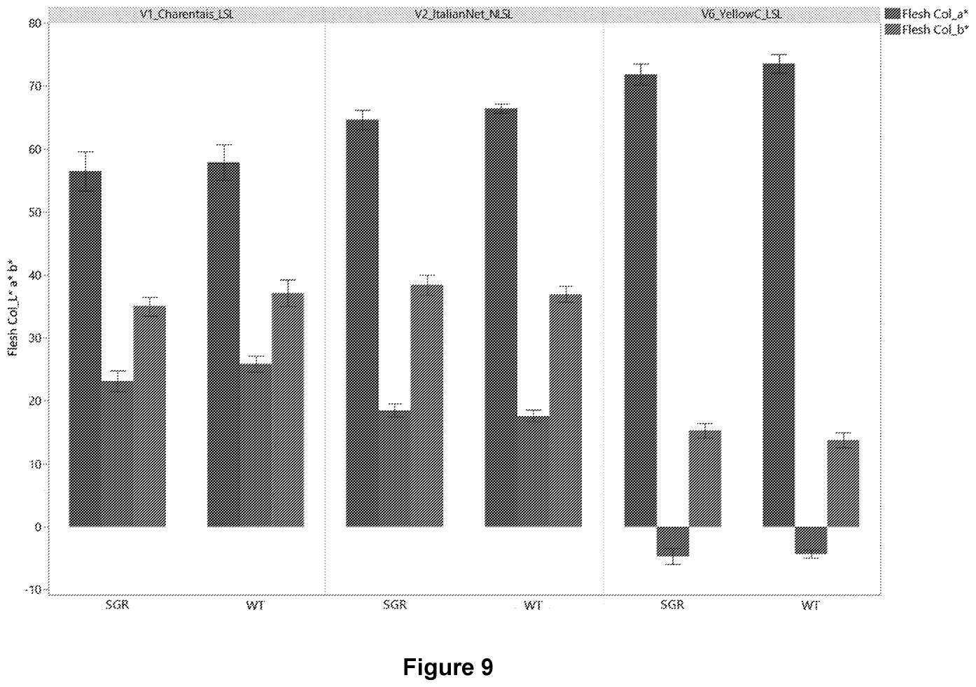

Color differences were assessed using pairwise comparison with Tukey's test and no significant differences were recorded between the sgr-1 lines and their WT versions in both orange flesh and white flesh material (FIG. 9).

Same results are observed on green-flesh materials.

### Example 6: Fine Phenotyping of sgr-1 Converted Varieties

A fine phenotyping of several traits was performed on 3 varieties comprising the WT allele, V2_ItalianNet_NLSL, V4_HD_NLSL, V5_Charentais_NLSL and their corresponding 3 converted varieties for the sgr-1 variation. A total of 14 fruits per genotype were harvested and phenotyped to assess the effects of the sgr-1 mutation on other important characteristics linked to Non LSL types.

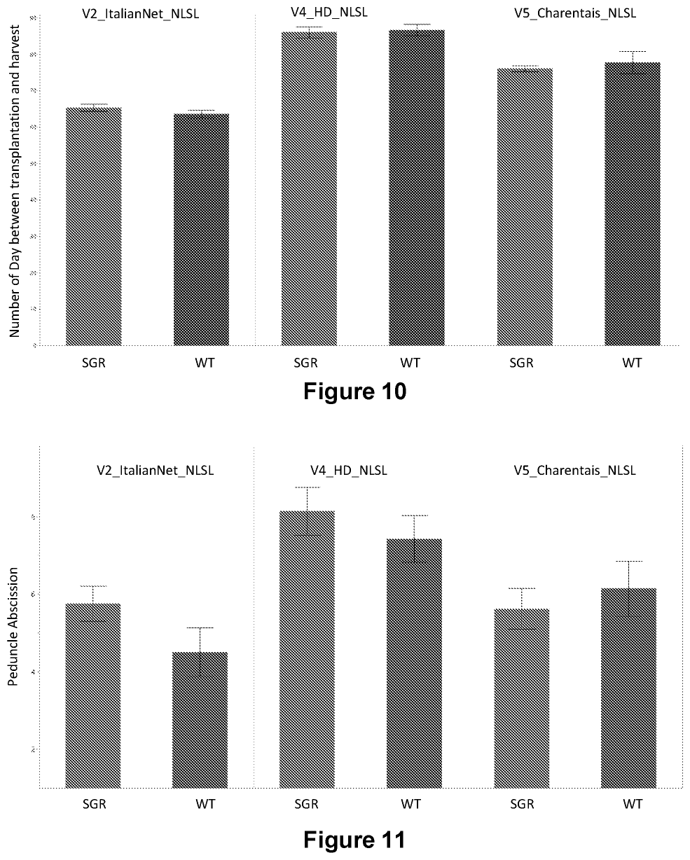

Cycle length corresponds to the number of days calculated between the transplanting date and the harvest date. Non-LSL material is known as the shortest cycles, fruits can be harvested from 55 days after transplantation, whereas LSL material has longer cycles, fruits can be harvested around 90 days after transplantation. In the experiment, all the plants were transplanted the same day in the field (around 20 days after the sowing) and each fruit harvested were recorded with its harvest date to make the calculation. In the different genotypes observed, there were no significant differences between the sgr-1 converted varieties and their corresponding WT varieties for the cycle length (FIG. 10). The stay green mutation does not delay significantly the harvest time.

Peduncle abscission is an important maturity indicator such as the rind color evolution or the first leaf and tendril senescence. Fruits are harvested when one or several of these indicators are moving. Therefore, peduncle abscission was observed the day of the fruit harvest, and assessed on a scale from 1 to 9, wherein 1=full sipping, 9=not slipping. The sgr-1 mutation does not impact too much the maturity indicator which is the peduncle abscission, no significant differences are found using pairwise comparison with Tukey's test (FIG. 11). Slipping material stays slipping and will ease the harvest by the growers.

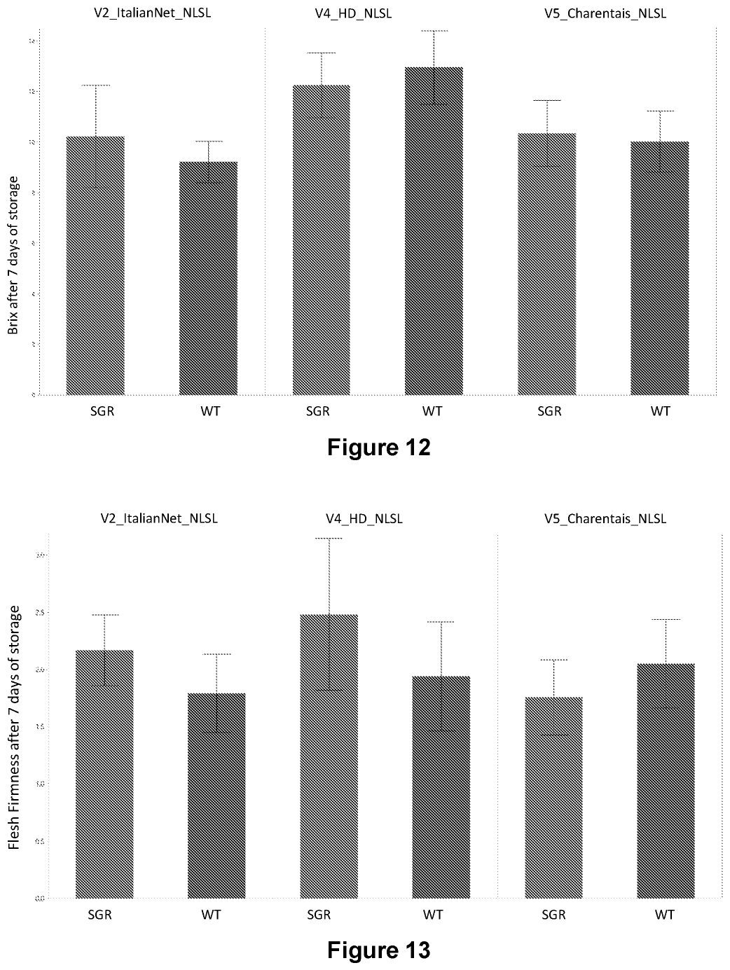

The brix measurements performed the harvest day on the equatorial slice of each melon with an electronic refractometer also show no significant difference of the brix level of the sgr-1 mutants in comparison to the WT versions. The sgr-1 mutation does not impact the sugar level, and thus sweetness, in comparison with the corresponding melons carrying the WT allele (FIG. 12).

Firmness was also measured on each equatorial slice of all the harvested fruits. The measurement was made using a penetrometer, on 2 diametrically opposed points of the equatorial slice. The mean of the two measurements is then calculated. No statistical difference could be observed between sgr-1 and WT genotypes (FIG. 13)

In conclusion, the observations done on different sgr-1 converted varieties in comparison to the original ones (WT) classified as non-LSL genotypes, show that the sgr-1 mutation introduces a new ideotypes of melons, with an extended shelf life thanks to a stability of the rind color evolution but without any impact on the fruit quality and maturity indicators. In other words, it will bring a better field holding and a fruit harvest flexibility to the growers, without extending the harvest windows. The non-evolution of the rind color during the storage brings more flexibility to the retailers. For the final consumer, the initial fruit qualities of the product are maintained.

Other climacteric fruits from different melon typologies have been assessed and the same conclusions are made.

1-17. (canceled)

18. A Cucumis melo plant, or a plant part or cell thereof, wherein said plant homozygously comprises in its genome a mutant allele of the staygreen (sgr) gene on chromosome 9, wherein said mutant allele of the sgr gene comprises at least one loss-of-function mutation in comparison to the sequence of a wild-type sgr allele (SEQ ID NO:1) and wherein said mutant allele of the sgr gene confers rind color stability to the fruits of said plant at maturity and/or during post-harvest, in comparison with an isogenic non-long shelf life (non-LSL) Cucumis melo plant which does not comprise said mutant allele.

19. The plant, plant part or cell according to claim 18, wherein said at least one loss-of-function mutation is selected from a nonsense mutation, a framework mutation or a defective splicing mutation.

20. The plant, plant part or cell according to claim 18, wherein said at least one loss-of-function mutation is a splicing site mutation.

21. The plant, plant part or cell according to claim 19, wherein said at least one loss-of-function mutation consists in a G584A mutation in the sequence set forth in SEQ ID NO:1.

22. The plant, plant part or cell according to claim 18, wherein the firmness at maturity, the degree of peduncle abscission at maturity and/or the cycle length of the fruits of said plant, is substantially unchanged, in comparison to the fruits of an isogenic plant at the same stage of maturity and grown in the same environmental condition, wherein said isogenic plant does not comprise homozygously in its genome said mutant allele of the sgr gene.

23. The plant, plant part or cell according to claim 18, wherein said C. melo plant is a plant from an inbred C. melo line or is an F1 hybrid C. melo plant.

24. The plant, plant part or cell according to claim 18, wherein the cell is derived from an embryo, protoplast, meristematic cell, callus, pollen, leaf, anther, stem, petiole, root, root tip, fruit, seed, flower, cotyledon, and/or hypocotyl, wherein said cell comprises in its genome a mutant allele of the staygreen (sgr) gene on chromosome 9, wherein said mutant allele of the sgr gene homozygously comprises at least one loss-of-function mutation in comparison to the sequence of a wild-type sgr allele (SEQ ID NO:1).

25. The plant, plant part or cell according to claim 18, wherein said plant part is an embryo, protoplast, meristematic cell, callus, pollen, leaf, anther, stem, petiole, root, root tip, fruit, seed, flower, cotyledon, or hypocotyl.

26. A C. melo seed, which can be grown into a C. melo plant according to claim 18.

27. A method of producing a C. melo plant producing fruits or susceptible to produce fruits with an increased shelf life, comprising:
(a) using a Cucumis melo plant according to claim 18 as a breeding partner to introgress the mutant allele mutant allele of the sgr gene into a progeny C. melo plant; or
(b) introducing by mutagenesis or genome editing a loss-of-function mutation in the sgr gene on chromosome 9 in the genome of a C. melo plant.

28. The method of claim 27, wherein using said Cucumis melo plant as a breeding partner to introgress the mutant allele mutant allele of the sgr gene into a progeny C. melo plant comprises:
(al) crossing said Cucumis melo plant with a second C. melo plant which does not homozygously comprise said mutant allele of the sgr gene, thus generating a F1 population,
(a2) advancing the F1 population to create F2 population,
(c) selecting one plant homozygously comprising said mutant allele in the progeny thus obtained.

29. The method of claim 28, further comprising self-pollinating one or several times the plant obtained at step c) and/or backcrossing the plant selected in step b) or c).

30. A method for identifying, detecting and/or selecting C. melo plants producing fruits or susceptible to produce fruits with an increased shelf life, said method comprising the detection of a mutant allele of the sgr gene on chromosome 9 in the genome of said plants, wherein said mutant allele comprises at least one loss-of-function mutation in comparison to the sequence of a wild-type sgr allele (SEQ ID NO:1).

31. The method according to claim 30, wherein said loss-of-function mutation is selected from a nonsense mutation, an indel mutation, a framework mutation or a defective splicing mutation.

32. The method according to claim 30, wherein said at least one loss-of-function mutation is a splicing site mutation.

33. The method according to claim 30, wherein said at least one loss-of-function mutation consists in a G584A mutation in the sequence set forth in SEQ ID NO:1.

34. The method according to claim 30, comprising the detection of the sequence set forth in SEQ ID NO:2.


SEQUENCE LISTING (8 sequences)

SEQ ID NO: 1 - DNA, 2480 bp, Cucumis melo
attgtagtgggtaggatcgaaagtcaaaaacagagacggtgtcgtattagtaatcaaccacgtaaacccctccaaaaagaaaaatggttgcttattacaatcaaacctaaaacaaagtctagtacttgaagcaacagcgaagaaaaaaagctattcctttccaacacgcttctaattcccaaaaattctggatttcaccactcttcaggattgtacacaacatttcctttttcattcttcccctttttctcctttttcttttttcttttgtttcctacaagcctcatatttcttcctcccctaagattcctctgtgtttttttatctttcaaaccgatcacaaaccctccctaatttctggagctaatctgttggagaagatgagggttttgactagtaattcttctcctcttcttgttccttcttcaaacccttatcaggattcttcttctctcttcctctgtaaacgcaaatccaaggagaaaaaccacagaatggttcctgtaagtctttttttttttttttcattaaagattgaactttcgaaagtgggttttgttttgattttgattttaattttatagatggcgagattgtttgggccagccatatttgaagcttcgaagctaaaggttctgtttttaggggtggatgagaagaaacatccaggcaaatttccaaggacttatacgcttacacatagtgatattacttctaaacttactcttgccatttctcaatccattaacaattctcaggtaactcttcttcttttgtttatttaatgaaaaccccttttttagtttatcagatgtttggaaattgtaaaaagagtaattgatctttgttttagaaatgattttgaaagttttgttttaggttataattatgtgaaacgtgtttttttattttcatcgtgcttttctttaccacttcaatcgatcttcaataatgcttctcatatcagataagtattgtttttttttttcttttttaatcattttatttagcttgaagacactacttcaatattaaaaaaaaaaaaagctacaatttcgaaaactataaaatagtttagaaaaatatgttttggttttagaattgggtaagggtacatttgagccctttttaggaataataaatttaaaatatttcaacatttttctataacgggaaatataataaaattcatgttttatggatttttataatcaataaatagccataaattttaagaactaaatctaaattttgttacatttacaaatatttttgagactatagtatatcttccaggtatgtttataattgcattttaaaatttggttagaaattcaagtttcttcaataggagtgaaaatcatagtatgaaattcgacaatgttactgaatagagagcttcgagtcccaacaatccagttggaaaattggaagataaaaatttactactatatgggatttttttttttttttttaattttttggtatgggtgttaaaaatgtaagtattgtaattgatgatggatgaataatatattatgcagttacaaggatggtataattggcttcaaagggatgaagtggtagcagaatggaagaaagtacaggggaaaatgtcccttcatgttcattgtcatatcagtggtggccattttcttctagatctctgtgctaatctccgatacttcatctttcgcagagaacttcctgtggtaatttctctctctttttcacaactacccatagtaaaatcaatcattgctatagtaaataaatttttacctatgggcgaattatttgcactatgtcaggtctatttgtccaatagaaaaactggatgtagtattgaaacatgtcccattttttgaattattgttattatataattgaacatatggttgacacgtgatgaaaaaaaataaaaataaaacaggtgctgaatgctttcgtccatggagatgtggacttgttcaagaattacccagagctacaagaggctatggtttgggtttatttccactccaaaattccggaattcaacaaagtagaatgctggggcccactaaaggatccagccccaccttcaagtgggcttgatgggcggcccaaatcagatgagcccatgtgggaattgagccggatggagcggcccaaaccttgccaagaagactgcaattgttgcttcccaaccatcccttccatttcatggtcccccaagaacagtgagttggagagcacgtgattgattttatttggctcccccttaaatgtaaatagttaatatcaatatttgttgttattatataatataacatccattcctattcctccaatgttttagtccttatattttaattcattaatctttatagtatctatgatgtgaaagtaggaaagtatcaaggaaatgttctttgatgaactataaccaaatttctccttatattttgatcaaatcaaatctttttggcttgtggttc

SEQ ID NO: 2 - DNA, 2480 bp, Cucumis melo
attgtagtgggtaggatcgaaagtcaaaaacagagacggtgtcgtattagtaatcaaccacgtaaacccctccaaaaagaaaaatggttgcttattacaatcaaacctaaaacaaagtctagtacttgaagcaacagcgaagaaaaaaagctattcctttccaacacgcttctaattcccaaaaattctggatttcaccactcttcaggattgtacacaacatttcctttttcattcttcccctttttctcctttttcttttttcttttgtttcctacaagcctcatatttcttcctcccctaagattcctctgtgtttttttatctttcaaaccgatcacaaaccctccctaatttctggagctaatctgttggagaagatgagggttttgactagtaattcttctcctcttcttgttccttcttcaaacccttatcaggattcttcttctctcttcctctgtaaacgcaaatccaaggagaaaaaccacagaatggttcctgtaagtctttttttttttttttcattaaagattgaactttcgaaagtgggttttgttttgattttgattttaattttataaatggcgagattgtttgggccagccatatttgaagcttcgaagctaaaggttctgtttttaggggtggatgagaagaaacatccaggcaaatttccaaggacttatacgcttacacatagtgatattacttctaaacttactcttgccatttctcaatccattaacaattctcaggtaactcttcttcttttgtttatttaatgaaaaccccttttttagtttatcagatgtttggaaattgtaaaaagagtaattgatctttgttttagaaatgattttgaaagttttgttttaggttataattatgtgaaacgtgtttttttattttcatcgtgcttttctttaccacttcaatcgatcttcaataatgcttctcatatcagataagtattgtttttttttttcttttttaatcattttatttagcttgaagacactacttcaatattaaaaaaaaaaaaagctacaatttcgaaaactataaaatagtttagaaaaatatgttttggttttagaattgggtaagggtacatttgagccctttttaggaataataaatttaaaatatttcaacatttttctataacgggaaatataataaaattcatgttttatggatttttataatcaataaatagccataaattttaagaactaaatctaaattttgttacatttacaaatatttttgagactatagtatatcttccaggtatgtttataattgcattttaaaatttggttagaaattcaagtttcttcaataggagtgaaaatcatagtatgaaattcgacaatgttactgaatagagagcttcgagtcccaacaatccagttggaaaattggaagataaaaatttactactatatgggatttttttttttttttttaattttttggtatgggtgttaaaaatgtaagtattgtaattgatgatggatgaataatatattatgcagttacaaggatggtataattggcttcaaagggatgaagtggtagcagaatggaagaaagtacaggggaaaatgtcccttcatgttcattgtcatatcagtggtggccattttcttctagatctctgtgctaatctccgatacttcatctttcgcagagaacttcctgtggtaatttctctctctttttcacaactacccatagtaaaatcaatcattgctatagtaaataaatttttacctatgggcgaattatttgcactatgtcaggtctatttgtccaatagaaaaactggatgtagtattgaaacatgtcccattttttgaattattgttattatataattgaacatatggttgacacgtgatgaaaaaaaataaaaataaaacaggtgctgaatgctttcgtccatggagatgtggacttgttcaagaattacccagagctacaagaggctatggtttgggtttatttccactccaaaattccggaattcaacaaagtagaatgctggggcccactaaaggatccagccccaccttcaagtgggcttgatgggcggcccaaatcagatgagcccatgtgggaattgagccggatggagcggcccaaaccttgccaagaagactgcaattgttgcttcccaaccatcccttccatttcatggtcccccaagaacagtgagttggagagcacgtgattgattttatttggctcccccttaaatgtaaatagttaatatcaatatttgttgttattatataatataacatccattcctattcctccaatgttttagtccttatattttaattcattaatctttatagtatctatgatgtgaaagtaggaaagtatcaaggaaatgttctttgatgaactataaccaaatttctccttatattttgatcaaatcaaatctttttggcttgtggttc

SEQ ID NO: 3 - DNA, 774 bp, Cucumis melo
atgagggttttgactagtaattcttctcctcttcttgttccttcttcaaacccttatcaggattcttcttctctcttcctctgtaaacgcaaatccaaggagaaaaaccacagaatggttcctatggcgagattgtttgggccagccatatttgaagcttcgaagctaaaggttctgtttttaggggtggatgagaagaaacatccaggcaaatttccaaggacttatacgcttacacatagtgatattacttctaaacttactcttgccatttctcaatccattaacaattctcagttacaaggatggtataattggcttcaaagggatgaagtggtagcagaatggaagaaagtacaggggaaaatgtcccttcatgttcattgtcatatcagtggtggccattttcttctagatctctgtgctaatctccgatacttcatctttcgcagagaacttcctgtggtgctgaatgctttcgtccatggagatgtggacttgttcaagaattacccagagctacaagaggctatggtttgggtttatttccactccaaaattccggaattcaacaaagtagaatgctggggcccactaaaggatccagccccaccttcaagtgggcttgatgggcggcccaaatcagatgagcccatgtgggaattgagccggatggagcggcccaaaccttgccaagaagactgcaattgttgcttcccaaccatcccttccatttcatggtcccccaagaacagtgagttggagagcacgtga

SEQ ID NO: 4 - protein, 257 aa, Cucumis melo
MRVLTSNSSPLLVPSSNPYQDSSSLFLCKRKSKEKNHRMVPMARLFGPAIFEASKLKVLFLGVDEKKHPGKFPRTYTLTHSDITSKLTLAISQSINNSQLQGWYNWLQRDEVVAEWKKVQGKMSLHVHCHISGGHFLLDLCANLRYFIFRRELPVVLNAFVHGDVDLFKNYPELQEAMVWVYFHSKIPEFNKVECWGPLKDPAPPSSGLDGRPKSDEPMWELSRMERPKPCQEDCNCCFPTIPSISWSPKNSELEST

SEQ ID NO: 5 - DNA, 364 bp, Cucumis melo
  misc_feature (144)..(144): n is a, c, g, or t
  Polymorphism (203)..(203): Wild-type=G;Mutant=A
tctgttggagaagatgagggttttgactagtaattcttctcctcttcttgttccttcttcaaacccttatcaggattcttcttctctcttcctctgtaaacgcaaatccaaggagaaaaaccacagaatggttcctgtaagtcncattaaagattgaactttcgaaagtgggttttgttttgattttgattttaattttataratggcgagattgtttgggccagccatatttgaagcttcgaagctaaaggttctgtttttaggggtggatgagaagaaacatccaggcaaatttccaaggacttatacgcttacacatagtgatattacttctaaacttactcttgccatttctcaatccat

SEQ ID NO: 6 - DNA, 42 bp, Artificial Sequence
  forward primer (wild-type allele)
gaaggtgaccaagttcatgctggcccaaacaatctcgccatc

SEQ ID NO: 7 - DNA, 44 bp, Artificial Sequence
  forward primer (sgr-1 mutant allele)
gaaggtcggagtcaacggattctggcccaaacaatctcgccatt

SEQ ID NO: 8 - DNA, 30 bp, Artificial Sequence
  common reverse primer
gaactttcgaaagtgggttttgttttgatt